(19) United States
(12) Patent Application Publication
(10) Pub. No.: US 2021/0347724 A1
(43) Pub. Date: Nov. 11, 2021

(54) METHOXYCHALCONE DERIVATIVES AND USES THEREOF

(71) Applicant: The Board of Trustees of the Leland Stanford Junior University, Stanford, CA (US)

(72) Inventors: Meghan A. Rice, Palo Alto, CA (US); Vineet Kumar, Palo Alto, CA (US); Dhanir Tailor, Palo Alto, CA (US); Sanjay V. Malhotra, Palo Alto, CA (US); Tanya Stoyanova, Palo Alto, CA (US)

(21) Appl. No.: 17/307,699

(22) Filed: May 4, 2021

Related U.S. Application Data
(60) Provisional application No. 63/023,031, filed on May 11, 2020.

(51) Int. Cl. C07C 205/06; A61P 35/00; A61K 47/06; A61K 9/00

(52) U.S. Cl. CPC C07C 205/06; A61K 9/0019; A61K 47/06; A61P 35/00

(57) ABSTRACT

Methoxychalone derivatives with anti-cancer activity are provided.

### BACKGROUND

Prostate cancer remains the most commonly diagnosed noncutaneous tumor among men. The prostate gland is dependent on androgens and thus many prostate cancer therapeutic strategies rely on inhibition of androgen receptor (AR) signaling axis. Such is the case of aggressive prostate cancer, which is treated with hormone-deprivation therapy resulting in temporary success, with most patients progressing to incurable, and often metastatic, castration-resistant prostate cancer (CRPC). CRPC is treated with second-generation anti-androgen therapies such as enzalutamide or abiraterone acetate, as well as chemotherapy, and in the case of non-metastatic disease, apalutamide or darolutamide; yet these therapeutics have only temporary benefit. Thus, developing new therapeutics for aggressive prostate cancer that are independent of hormone and androgen signaling may reduce the progression of patients to castration resistance. Further, as CRPC remains incurable, there is critical need for new therapeutic strategies to treat CRPC.

Chalcones are one of the major classes of widely occurring natural products, which are intermediates in plant flavonoid/isoflavonoid synthesis. They are characterized by an α,β-unsaturated carbonyl structure with two aromatic rings and commonly act as radical scavengers. Previous studies demonstrated that chalcones can lead to induction of cell cycle arrest or apoptosis. Further development is of interest.

### SUMMARY

Methoxychalcone derivatives and methods of use thereof are provided. The compounds are demonstrated herein to have potent anticancer activity against multiple types of cancer, including without limitation prostate cancer. The compounds also are shown to have synergistic activity in combination with other anti-cancer agents.

In some embodiments a pharmaceutical formulation is provided, comprising a methoxychalcone derivative; and a pharmaceutically effective excipient. In some embodiments the formulation is provided in a unit dose form. In some embodiments the formulation is provided in sterile packaging for clinical use. In some embodiments the methoxychalcone derivative is a compound of Formula I or a derivative or pro-drug thereof, including without limitation

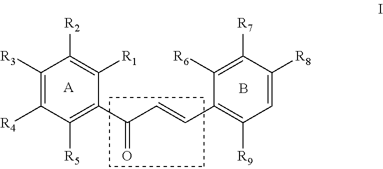

wherein:

R₇ is NO₂; and

each of R₁, R₂, R₃, R₄, R₅, R₆, R₈ and R₉ are independently selected from H, a lower alkyl, an alkoxy group with a lower alkyl; and the like, where a lower alkyl is meant alkyl groups containing from 1 to 6 carbon atoms, usually containing from 1-4 carbon atoms.

In some embodiments the methoxychalcone derivative is SU086:

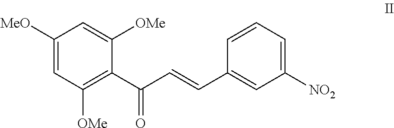 [SMILES: COc1cc(OC)c(C(=O)/C=C/c2cccc([N+](=O)[O-])c2)c(OC)c1]

In some embodiments, methods are provided for the treatment of cancer, the methods comprising administering an effective dose of a methoxychalcone derivative, for example a compound of Formula I or a derivative or prodrug thereof to an individual in need thereof. In some such embodiments the compound is SU086. In some embodiments the compound is delivered in combination with one or more additional anti-cancer treatments, where the combination may be additive or synergistic. In some embodiments the cancer is a solid cancer, e.g. a carcinoma. In some embodiments the cancer is prostate cancer. In some embodiments, prostate cancer is treated with a combination of SU086 and an anti-androgen compound, including without limitation enzalutamide, abiraterone, and the like.

Without being limited by the theory, it is believed that the anti-cancer activity of the present compounds is provided by inhibition of glycolytic metabolism. Combinations of the present compounds with agents that act on pathways other than inhibition of glycolytic pathways is of interest.

These and other advantages and features of the disclosure will become apparent to those persons skilled in the art upon reading the details of the compositions and methods of use, which are more fully described below.

### BRIEF DESCRIPTION OF THE FIGURES

The invention is best understood from the following detailed description when read in conjunction with the accompanying figures. It is emphasized that, according to common practice, the various features of the figures are not to-scale. On the contrary, the dimensions of the various features are arbitrarily expanded or reduced for clarity. Included in the drawings are the following figures. It is understood that the figures, described below, are for illustration purposes only. The figures are not intended to limit the scope of the present teachings in any way.

FIG. 1A-1D. Screening of chalcone analogues and selection of an anti-prostate cancer treatment FIG. 1A. Structures of twenty-two chalcone analogues used in screening against the NCI-60 panel of sixty cancer cell lines. FIG. 1B. Heat map of chalcone library assay on DU145 and PC3 cells from NCI-60 cell line panel. Each cell line was treated with indicated chalcone for 48 hours at preliminary concentration of 10 μM. Growth inhibition was then measured using sulforhodamine B assay per standard NCI protocol. Growth inhibition is indicated in a gradient from blue (no growth inhibition) to red (complete growth inhibition). FIG. 1C. Structure of SU086.

FIG. 1D. IC₅₀ determination by cell titer blue viability assay in C4-2 cells treated for 48 hours with concentrations of SU086 from 0.0625 to 10 μM. Error bars indicate ±SD.

FIG. 2A-2E. SU086 inhibits prostate cancer cell growth, migration and invasion in vitro. FIG. 2A. Proliferation of C4-2, DU145 and 22RV1 cells over 6 days in presence of 1 μM SU086 or vehicle control measured by cell number via trypan blue exclusion assay. FIG. 2B. Colony formation assay of C4-2, DU145 or 22RV1 cells grown 9 days in presence of 1 μM SU086 or vehicle control, graphed as colony formation rate (colony number/500 cells plated×100). Scale bar=100 mm. FIG. 2C. LuCaP 136 and LuCaP 147 cells grown as tumorspheres in presence of 1 μM SU086 or vehicle control. Graph represents area of well covered by spheres as an average of three wells ±SD. Scale bar (top)=300 microns; enlarged=50 microns FIG. 2D. Migration assay of C4-2, DU145 and 22RV1 cells following 72 hours pretreatment with 1 μM SU086 or DMSO control. After pretreatment, viable cells were counted and the same number were plated and cultured in transwell chambers for 20 hours, with continued treatment of SU086 or DMSO control in supplemented and unsupplemented media. Cells per field are graphed. Scale bar=250 microns. FIG. 2E. Matrigel dot assay invasion assay of C4-2 and DU145 cells in presence of 1 μM SU086 or vehicle control measuring distance migrated after escape from Matrigel dot, graphed as distance (mm). Scale bar=250 microns. All experiments were performed in triplicate with triplicate wells. Representative experiments are shown. For all, error bars represent standard deviation. *=P<0.05, **=P<0.01, ***=P<0.005, ****=P<0.001 determined by student's t-test.

FIG. 3A-3B. SU086 inhibits prostate cancer growth in pre-clinical models of prostate cancer. C4-2 FIG. 3A. or DU145 FIG. 3B. xenografts were established in NSG mice by implanting 5×10⁵ cells s.c. in 50 μl of Matrigel. Treatment with SU086 (50 mg/kg i.p. daily) or vehicle control was initiated when average tumor volume reached 50-mm³. Animal weights and tumor volumes (L×W×H/2) were measured every third day. Tumors were graphed as fold change over respective Day 0 volumes±SEM (C4-2: DMSO n=10, SU086 n=9; DU145: DMSO n=10, SU086 n=8). Tumors were stained for H&E and Ki67. Ki67 proliferative index was quantified as percentage positive nuclei per field, as an average of three fields, SD. Scale bars indicate 25 μm. *=P<0.05, **=P<0.01, ****=P<0.001 determined by student's t-test.

FIG. 4A-4D. SU086 inhibits prostate cancer growth of PDX models of prostate cancer and ex vivo prostate cancer specimens. FIG. 4A. Schematic diagram of PDX models. Xenografts were generated by implantation of cultured LuCaP 147 or LuCaP 136 cells into the rear flank of immunocompromised NSG mice concurrently with testosterone pellet in a remote s.c. implantation site. 5×10⁶ cells were implanted and grown one month. Tumor chunks (25 mg) were serially passaged. Transplanted tumors were grown to 50 mm³ volume prior to randomization and treatment initiation with 50 mg/kg SU086 or vehicle control. Animal weights and tumor volumes (L×W×H/2) were measured every third day. After day 18 tumors were harvested. FIG. 4B, FIG. 4C. LuCaP 147 and LuCaP 136 tumor volumes are graphed as fold-change over Day 0±SEM. Accompanying histological analysis of subcutaneous xenografts and Ki67 staining was performed. Ki67 proliferative index was quantified as percentage positive nuclei per field, as an average of three fields, SD. Scale bars indicate 25 microns. FIG. 4D. Ex vivo culture of human prostate cancer in medium with vehicle or SU086 (5 μM) treatment, three slices per treatment. Tissues was cultured in a rotating apparatus for 72 hours with media and compound exchanged daily. Specimens were assayed by H&E and Ki67 staining as described above, graphed±SD. *=P<0.05, **=P<0.01, ns=no significance. Scale bars are 50 microns.

FIG. 5A-5I. Proteomic analysis identifies the effect of SU086 on glycolysis impairment. FIG. 5A. C4-2 and DU145 cells treated 48 hours with 1 μM SU086 or DMSO control were harvested, lysed, and digested with trypsin. Liquid chromatography tandem mass spectrometry (LC-MS/MS) was performed. Cutoff values of 2-fold change with a FDR of <1% are included. Heatmap indicates −7 to +7 z score of MS1 data. FIG. 5B. z score graphed as function of p value (P<0.01) in volcano plot for C4-2 and DU145 proteomics results. Blue indicates decreased fold-change and green indicates increased change. FIG. 5C. Significantly down-regulated proteins were compared between cell lines, with 29 identified as overlapping. FIG. 5D. These common significantly down-regulated proteins were graphed in String, enriching for glycolysis (red filled circles). e. IHC validation of target PGK1 in SU086 treated xenograft and PDX specimens. Scale bars=25 microns. FIG. 5F, FIG. 5G. Glycolytic challenge assay performed on Seahorse XF of C4-2 (FIG. 5F) and DU145 (FIG. 5G.) cells pretreated 24 hours with 1 μM SU086 or DMSO. ECAR-extracellular acidification (mpH/min) indicates glycolysis. Flux cartridge injects glucose, Oligomycin (oligo) and 2-DG at indicated points to measure metabolic initiation, peak glycolytic capacity, and depleted levels respectively, and graphed±SD. The sixth timepoint minus the third was quantified as glycolytic flux, while the ninth minus the third timepoint was quantified as glycolytic capacity. DESI-MS analysis of OCT-embedded C4-2 (FIG. 5H)(n=2 per condition) and DU145 (FIG. 5I) (n=6 per condition) xenografts treated with SU086 or vehicle control from FIG. 2, indicating localization of lactate and pyruvate levels in the tumors. DESI analyzed tissue was histologically analyzed and regions of non-necrotic tumor (outlined in black) were digitally pixelated and quantitatively analyzed using SAM analysis. Quantified lactate/pyruvate ratios across all pixels are graphed as violin plots with horizontal lines indicating median and quartiles. *=P<0.05, **=P<0.01.

FIG. 6A-6E. SU086 synergizes with enzalutamide and abiraterone acetate to decrease prostate cancer growth in vitro and in vivo. FIG. 6A. Coefficient of Drug Interaction (CDI) was tested with colony formation data of each compound at varied single dose concentrations and combination concentrations-SU086 (250 nM-1 μM), Enzalutamide (ENZ 1 μM-5 μM), Abiraterone acetate (ABI 500 nM-2.5 μM). CDI=AB (SU086+ENZ treatment, or SU086+ABI treatment)/[(A, SU086 treatment alone)×(B, ENZ or ABI treatment alone)]. Values less than one indicate synergism, values equal to one indicate additive effects, and values greater than one indicate antagonism. Final concentrations were calculated with the final dosing as noted, SU086 (250 nM), ENZ (5 μM), ABI (2.5 μM). As such, SU086 synergizes with ENZ and has additive effects with ABI. FIG. 6B. Colony formation of C4-2 cells with SU086 (250 nM), ENZ (5 μM), ABI (2.5 μM), SU086+ENZ, SU086+ABI, or DMSO control. Representative well images are shown, scale bar=100 mm. FIG. 6C. Tumorsphere growth of LuCaP 136 cells embedded in 50% Matrigel in the presence of SU086 (1 μM) plus ENZ (5 μM) or ABI (2.5 μM) for 15 days. Wells were scanned with Celigo Imager and whole wells counted manually from images in triplicate. Scale bar (top)=300 microns; enlarged=50 microns. In vitro assays performed in triplicate with representative image shown, graphed±SD. FIG. 6D. C4-2 xenografts of mice treated with vehicle control, SU086 (50 mg/kg daily i.p.), ENZ (10 mg/kg daily gavage), ABI (200 mg/kg daily gavage), or SU086 plus ENZ or ABI. Mice were treated 21 days with tumors measured every third day. Tumors graphed as fold-change over Day 0 per each individual tumor. Graphed±SEM. FIG. 6E. H&E and Ki67 IHC of tumors from combination therapy. Ki67 proliferative index was quantified by manual counting percent positive nuclei per image field, averaged for three separate fields and graphed±SD. Scale bars=50 μm. *=P<0.05; **=P<0.01; ***=P<0.005; ****=P<0.001.

FIG. 7. NCI-60 cell line panel. Twenty-two chalcone analogues were assayed against the NCI-60 cell line panel at 10 μM treatment for 48 hours. Growth inhibition was measured and displayed as a heatmap from no inhibition (blue), to complete growth inhibition (red). White indicates no measurements were acquired.

FIG. 8. Pharmacokinetic analysis of SU086. Mice were treated with 50 mg/kg SU086 i.p. at time zero. Blood was collected retro-orbitally at 5, 15, 30, 60, 120, 360 and 720 minutes after injection in three mice per time point. Plasma concentration of SU086 was detected at the indicated concentrations and graphed as μg/μl±SEM. Cₘₐₓ (maximum serum concentration), Tₘₐₓ(time to reach Cmax) and T_{1/2} (elimination half-life of compound) are indicated. Values are then defined on a table calculating final concentration (μM) of SU086 in treated plasma.

FIG. 9A-9D. SU086 assayed toxicity from single therapy. FIG. 9A. Liver enzyme panel was performed on mice treated with SU086 or vehicle control (three mice per condition). Liver enzymes tested include Aspartate Transaminase (AST), Alanine Aminotransferase (ALT), Alkaline Phosphatase (ALP), Gamma-Glutamyl Transferase (GGT), and Total Bilirubin. Samples are charted as average of Units/Liter (U/L)±SEM. Statistical analysis: P Values were calculated with Student's T-test using the U/L data. Samples were further charted over the upper limit of normal (ULN) by dividing by the following values: AST (388 U/L), ALT (160 U/L), ALP (183). GGT and Bilirubin are not often expressed in normal samples and therefore do not have a ULN value. FIG. 9B. Animal weight and tumor volume (L×W×H/2) were measured every third day from experiment in FIG. 2a,b. b. Animal weight and tumor volume (L×W×H/2) were measured every third day from experiment in FIG. 3b,c. FIG. 9C. FIG. 9D. Liver and kidney tissues were harvested from SU086 treatment study. Fresh samples were imaged on brightfield on stereomicroscope. Scale bar represents 1 mM. Tissue was subsequently fixed in 10% formalin overnight at 4° C., processed and embedded. Samples were then H&E stained. Scale bars=500 microns left, 50 microns enlarged.

FIG. 10A-10C. Early effects of glycolysis. FIG. 10A. Proliferation of C4-2 and DU145 cells at 1, 2, 3 days treatment with 1 μM SU086 or vehicle control and counted by trypan blue exclusion assay. FIG. 10B, FIG. 10C. OCR (oxygen consumption rates) of 24-hour Seahorse glycolytic stress test for C4-2 and DU145 cells reflecting no changes. OCR levels (y-axis) were than graphed against ECAR levels (x-axis) for 24-hour treatment SU086 versus DMSO cells. Samples are the average of three wells, with up-down error bars representing OCR standard deviation, and left-right error bars indicating ECAR standard deviation. *=P<0.05, **=P<0.01, ns=no significance.

FIG. 11A-11C. DESI analysis. FIG. 11A. Desorption electrospray ionization (DESI) experimental workflow in hivh solvent is ionized and sprayed onto C4-2 or DU145 xengoraft tissues from FIG. 2 which had undergone in vivo treatment of SU086 at 50 mg/kg or DMSO vehicle control. Charged droplets containing metabolites from tissues were then picked up by MS reflecting coordinately mapped metabolites across tissues. Samples were then ran through LC/MS-MS analysis as described. FIG. 11B. Profiles of all C4-2 and DU145 spectral analyses unique pixels. FIG. 11C. Metabolite spectra for a representative row of one tissue sample. Commonly occurring metabolites are marked in red reflecting m/z size. C4-2 n=2 xenografts per condition; DU145 n=6 xenografts per condition.

FIG. 12A-12B. In vivo animal weights from FIG. 4. FIG. 12A. Mice from FIG. 4D were weighed every third day to assay toxicity as a result of combination therapy of SU086 with second-generation antiandrogens enzalutamide (ENZ) or abiraterone (ABI). FIG. 12B. At sacrifice, liver and kidneys were isolated and imaged on brightfield. Scale bar represents 1 mM. Tissue was subsequently fixed in 10% formalin overnight at 4° C., processed and embedded. Samples were then H&E stained. Scale bars=500 microns left, 50 microns enlarged.

FIG. 13A-13B. Graphical representation of findings. FIG. 13A. Overview of effects of SU086 in vitro (blue), in vivo (orange), human samples (green) and overall findings (purple). FIG. 13B. Schematic of proposed model of SU086 effect on prostate cancer glycolytic switching.

### DEFINITIONS

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction of tumor burden).

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias.

In some embodiments a cancer treated with the methods described herein is a solid tumor, including particularly carcinomas. Examples of solid tumors include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-0-, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

As used herein, “carbocycle” or “carbocyclic ring” is intended to mean any stable monocyclic, bicyclic, or tricyclic ring having the specified number of carbons, any of which may be saturated, unsaturated, or aromatic. For example a C3-14 carbocycle is intended to mean a mono-, bi-, or tricyclic ring having 3, 4, 5, 6, 7, 8, 9, 10, 11, 12, 13, or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when a covalent bond or one or more carbon atoms link two non-adjacent carbon atoms in a ring. In one embodiment, bridge rings are one or two carbon atoms. It is noted that a bridge always converts a monocyclic ring into a bicyclic ring. When a ring is bridged, the substituents recited for the ring may also be present on the bridge. Fused (e.g., naphthyl and tetrahydronaphthyl) and spiro rings are also included.

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” refers to an aromatic group of from 1 to 15 carbon atoms, such as from 1 to 10 carbon atoms and 1 to 10 heteroatoms selected from oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

As used herein, the terms “Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycle and heteroaryls include, but are not limited to, 1,3-dioxolane, 1,4-dioxane, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N+≡C—), cyanato (—O—C≡N), isocyanato (—O—N+≡C—), isothiocyanato (—S—C≡N), azido (—N═N+≡N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻ M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻ M⁺, —P(O)(OR⁷⁰) 2, —C(O)R⁷⁰, —C(S)R⁷⁰, —C(NR⁷⁰)R⁷⁰, —C(O)O⁻ M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as +N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻ M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻ M⁺, —P(O)(OR⁷⁰)₂, —C(O)R⁷⁰, —C(S)R⁷⁰, —C(NR⁷⁰)R⁷⁰, —CO₂⁻ M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻ M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻ M⁺-NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻ M⁺, —OS(O)₂OR⁷⁰, —P(O)(O⁻)₂(M⁺)₂, —P(O)(OR⁷⁰)⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O) NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compositions and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Definitions of other terms and concepts appear throughout the detailed description below.

### DETAILED DESCRIPTION OF THE EMBODIMENTS

As summarized above, aspects of the present disclosure include compounds, compositions and methods for treatment of cancer by administering an effective amount of a methoxychalcone derivative, for example a compound of Formula I, including without limitation SU086, or a derivative or prodrug thereof to an individual in need thereof.

Also provided are methods for treating cancer. Aspects of the methods include administering to a subject an effective amount of a methoxychalcone derivative, for example a compound of Formula I, including without limitation SU086, or a derivative or prodrug thereof to treat the subject for cancer. In certain aspects, the a methoxychalcone derivative, for example a compound of Formula I, including without limitation SU086, or a derivative or prodrug thereof is administered in combination with another active agent (e.g. a chemotherapeutic agent) and/or radiation therapy. In some cases the cancer is a solid tumor. In some cases the solid tumor is a carcinoma. In some cases a cancer is selected from ovarian carcinomas (OC); colorectal cancer, e.g. adenocarcinomas; prostate cancer, e.g. adenocarcinoma of the prostate; head and neck cancer (HNC), e.g. squamous cell carcinomas; lung cancer, e.g. small cell lung cancer, non-small cell lung cancers such as squamous cell carcinoma, adenocarcinoma, and large cell carcinoma, etc.; and breast cancer, e.g. ductal carcinoma in situ, invasive ductal carcinoma, invasive lobular carcinoma; and the like. In certain embodiments the cancer is prostate cancer. In some embodiments the cancer is advanced prostate cancer. In some embodiments the cancer is metastatic, e.g. metastatis prostate cancer.

Aspects of the present disclosure include a methoxychalcone derivative compound, for example a compound of Formula I, including without limitation SU086, or a derivative or prodrug thereof (e.g., as described herein), salts thereof (e.g., pharmaceutically acceptable salts), and/or solvate, hydrate and/or prodrug forms thereof.

It is understood that, in any compound described herein having one or more chiral centers, if an absolute stereochemistry is not expressly indicated, then each center may independently be of R-configuration or S-configuration or a mixture thereof. More specifically, where compounds described herein contain one or more chiral centers and/or double-bond isomers (i.e., geometric isomers), enantiomers or diastereomers, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds.

The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure. It will be appreciated that all permutations of salts, solvates, hydrates, prodrugs and stereoisomers are meant to be encompassed by the present disclosure.

In some embodiments, the subject compounds, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, p-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)). In some cases, the promoiety is attached to the nitro group of the subject compounds. In certain cases, the promoiety is an acyl or substituted acyl group. In certain cases, the promoiety is an alkyl or substituted alkyl group, e.g., that forms an ester functional group when attached to the subject compounds.

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the subject compounds are provided by oral dosing and absorbed into the bloodstream. In some embodiments, the oral bioavailability of the subject compounds is 30% or more. Modifications may be made to the subject compounds or their formulations using any convenient methods to increase absorption across the gut lumen or their bioavailability.

In some embodiments, the subject compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

### Methods of Treatment

Aspects of the methods include administering to a subject an effective amount of a methoxychalcone derivative, for example a compound of Formula I, including without limitation SU086, or a derivative or prodrug thereof to treat the subject for cancer.

In some embodiments, an “effective amount” is an amount of a subject compound that, when administered to an individual in one or more doses, in monotherapy or in combination therapy, is effective to decrease tumor burden in the subject by about 20%, at least about 30%, at least about 40%, at least about 50%, at least about 60%, at least about 70%, at least about 80%, or at least about 90%, compared to tumor burden in the individual in the absence of treatment with the compound, or alternatively, compared to the tumor burden in the subject before or after treatment with the compound. As used herein the term “tumor burden” refers to the total mass of tumor tissue carried by a subject with cancer.

In some embodiments, an “effective amount” is an amount of a subject compound that, when administered to an individual in one or more doses, in monotherapy or in combination therapy, is effective to reduce the dose of conventional therapy required to observe tumor shrinkage in the subject by about 20%, at least about 30%, at least about 40%, at least about 50%, at least about 60%, at least about 70%, at least about 80%, or at least about 90%, compared to the dose of conventional therapy required to observe tumor shrinkage in the individual in the absence of treatment with the compound.

In some embodiments, an “effective amount” of a compound is an amount that, when administered in one or more doses to an individual having cancer, is effective to achieve a 1.5-log, a 2-log, a 2.5-log, a 3-log, a 3.5-log, a 4-log, a 4.5-log, or a 5-log reduction in tumor size.

In some embodiments, an effective amount of a compound is an amount that ranges from about 50 ng/kg body weight to about 50 μg/kg body weight (e.g., from about 50 ng/kg body weight to about 40 μg/kg body weight, from about 30 ng/kg body weight to about 20 μg/kg body weight, from about 50 ng/kg body weight to about 10 μg/kg body weight, from about 50 ng/kg body weight to about 1 μg/kg body weight, from about 50 ng/kg body weight to about 800 ng/kg body weight, from about 50 ng/kg body weight to about 700 ng/kg body weight, from about 50 ng/kg body weight to about 600 ng/kg body weight, from about 50 ng/kg body weight to about 500 ng/kg body weight, from about 50 ng/kg body weight to about 400 ng/kg body weight, from about 60 ng/kg body weight to about 400 ng/kg body weight, from about 70 ng/kg body weight to about 300 ng/kg body weight, from about 60 ng/kg body weight to about 100 ng/kg body weight, from about 65 ng/kg body weight to about 85 ng/kg body weight, from about 70 ng/kg body weight to about 90 ng/kg body weight, from about 200 ng/kg body weight to about 900 ng/kg body weight, from about 200 ng/kg body weight to about 800 ng/kg body weight, from about 200 ng/kg body weight to about 700 ng/kg body weight, from about 200 ng/kg body weight to about 600 ng/kg body weight, from about 200 ng/kg body weight to about 500 ng/kg body weight, from about 200 ng/kg body weight to about 400 ng/kg body weight, or from about 200 ng/kg body weight to about 300 ng/kg body weight).

In some embodiments, an effective amount of a compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 pg, from about 1 pg to about 10 pg, from about 10 pg to about 50 pg, from about 50 pg to about 150 pg, from about 150 pg to about 250 pg, from about 250 pg to about 500 pg, from about 500 pg to about 750 pg, from about 750 pg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Administration of an effective amount of a subject compound to an individual with cancer can result in one or more of: 1) a reduction in tumor burden; 2) a reduction in the dose of chemotherapy or radiotherapy required to effect tumor shrinkage; 3) a reduction in the spread of a cancer from one cell to another cell in an individual; 4) a reduction of morbidity or mortality in clinical outcomes; 5) shortening the total length of treatment when combined with other anti-cancer agents (e.g. resulting from sensitization to other anti-cancer agents); and 6) an improvement in an indicator of disease response (e.g., a reduction in one or more symptoms of cancer). Any of a variety of methods can be used to determine whether a treatment method is effective. For example, a biological sample obtained from an individual who has been treated with a subject method can be assayed.

Any of the compounds described herein can be utilized in the subject methods of treatment.

In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). The subject may be in need of treatment for cancer. In some instances, the subject methods include diagnosing cancer, including any one of the cancers described herein. In some embodiments, the compound is administered as a pharmaceutical preparation.

### Combination Therapies

The subject compounds can be administered to a subject alone or in combination with an additional, i.e., second, active agent. Combination therapeutic methods may include a combination with a second active agent or an additional therapy, e.g., radiation therapy. The terms “agent,” “compound,” and “drug” are used interchangeably herein. For example, compounds can be administered alone or in conjunction with one or more other drugs, such as drugs employed in the treatment of diseases of interest, including but not limited to, cancer. In some embodiments, the subject method further includes coadministering concomitantly or in sequence a second agent, e.g., a small molecule, a chemotherapeutic, an antibody, an antibody fragment, an antibody-drug conjugate, an aptamer, or a protein. In certain embodiments the second agent is a chemotherapeutic agent, e.g. an anti-androgen agent.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug or additional therapy with a pharmaceutical composition of the present disclosure means administration of the compound and second agent or additional therapy at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs or therapies and compounds of the present disclosure.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

In conjunction with any of the subject methods, the compounds (e.g., as described herein) (or pharmaceutical compositions comprising such compounds) can be administered in combination with another drug designed to treat a cancer. In certain cases, the compound can be administered prior to, at the same time as, or after the administration of the other drug. In certain cases, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioma, glioblastomas, melanoma and various head and neck tumors. In certain cases, the cancer is prostate cancer.

In some embodiments the combination comprises an anti-androgen drug. Antiandrogens, also known as androgen antagonists or testosterone blockers, are a class of drugs that prevent androgens like testosterone and dihydrotestosterone (DHT) from mediating their biological effects in the body. They act by blocking the androgen receptor (AR) and/or inhibiting or suppressing androgen production. Included in this class are AR antagonists, androgen synthesis inhibitors, and antigonadotropins. AR antagonists work by directly blocking the effects of androgens, while androgen synthesis inhibitors and antigonadotropins work by lowering androgen levels. AR antagonists can be further divided into steroidal antiandrogens and nonsteroidal antiandrogens; androgen synthesis inhibitors can be further divided mostly into CYP17A1 inhibitors and 5α-reductase inhibitors; and antigonadotropins can be further divided into gonadotropin-releasing hormone modulators (GnRH modulators), progestogens, and estrogens.

Useful anti-androgens for the methods of combination therapy of the present disclosure include, for example, enzalutamide, apalutamide, abiraterone, which are used in treatment of castration resistant prostate cancer. Other useful anti-androgens for combination therapy include, without limitation, steroidal antiandrogens, e.g. cyproterone acetate, megestrol acetate, chlormadinone acetate, spironolactone, oxendolone, and osaterone acetate; nonsteroidal antiandrogens, e.g. flutamide, bicalutamide, nilutamide, topilutamide, enzalutamide, and apalutamide; inhibitors of androgen synthesus, e.g. ketoconazole, abiraterone acetate, and seviteronel; CYP11A1 inhibitor aminoglutethimide; 5α-reductase inhibitors, e.g. finasteride, dutasteride, epristeride, alfatradiol; and other antiandrogens, e.g. cyproterone acetate, spironolactone, medrogestone, flutamide, nilutamide, bifluranol, bicalutamide flutamide; GnRH modulators, e.g. leuprorelin, cetrorelix, allylestrenol, chlormadinone acetate, cyproterone acetate, gestonorone caproate, hydroxyprogesterone caproate, medroxyprogesterone acetate, megestrol acetate, osaterone acetate, and oxendolone.

For the treatment of cancer, the compounds can be administered in combination with a chemotherapeutic agent selected from alkylating agents, nitrosoureas, antimetabolites, antitumor antibiotics, plant (vinca) alkaloids, steroid hormones, taxanes, nucleoside analogs, steroids, anthracyclines, thyroid hormone replacement drugs, thymidylate-targeted drugs, Chimeric Antigen Receptor/T cell therapies, Chimeric Antigen Receptor/NK cell therapies, apoptosis regulator inhibitors (e.g., B cell CLL/lymphoma 2 (BCL-2) BCL-2-like 1 (BCL-XL) inhibitors), CARP-1/CCAR1 (Cell division cycle and apoptosis regulator 1) inhibitors, colony-stimulating factor-1 receptor (CSF1R) inhibitors, CD47 inhibitors, cancer vaccine (e.g., a Th17-inducing dendritic cell vaccine, or a genetically modified tyrosinase such as Oncept®) and other cell therapies.

Specific chemotherapeutic agents of interest include, but are not limited to, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, and ABT-199. Peptidic compounds can also be used. Cancer chemotherapeutic agents of interest include, but are not limited to, taxane and active analogs and derivatives thereof. As used herein, the term “taxane” refers to compounds that have the basic taxane skeleton as a common structure feature. In certain embodiments, the taxane is paclitaxel. Paclitaxel is a highly derivatized diterpenoid (Wani, et al. (1971) J. Am. Chem. Soc. 93:2325-2327) which has been obtained from the harvested and dried bark of Taxus brevifolia (Pacific Yew) and Taxomyces andreanae, an endophytic fungus of the Pacific Yew (Stierle, et al. (1993) Science 60:214-216). Also included in the term “taxanes” are paclitaxel analogues, formulations, and derivatives, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel. As such, the term taxane refers to not only the common chemically available form of paclitaxel, but analogs (e.g., taxotere, as noted above) and paclitaxel conjugates (e.g., paclitaxel-PEG, paclitaxel-dextran, or paclitaxel-xylose). Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Any convenient cancer vaccine therapies and agents can be used in combination with the subject compounds, compositions and methods. For treatment of cancer, e.g., prostate cancer, the compounds can be administered in combination with a vaccination therapy, e.g., a dendritic cell (DC) vaccination agent that promotes Th1/Th17 immunity.

In certain instances, the combination provides an enhanced effect relative to either component alone; in some cases, the combination provides a supra-additive or synergistic effect relative to the combined or additive effects of the components. A variety of combinations of the subject compounds and the chemotherapeutic agent may be employed, used either sequentially or simultaneously. For multiple dosages, the two agents may directly alternate, or two or more doses of one agent may be alternated with a single dose of the other agent, for example. Simultaneous administration of both agents may also be alternated or otherwise interspersed with dosages of the individual agents. In some cases, the time between dosages may be for a period from about 1-6 hours, to about 6-12 hours, to about 12-24 hours, to about 1-2 days, to about 1-2 week or longer following the initiation of treatment.

### Utility

The compounds and methods of the invention, e.g., as described herein, find use in a variety of applications. Applications of interest include, but are not limited to: research applications and therapeutic applications. The subject compounds and methods may be used in the optimization of the bioavailability and metabolic stability of compounds.

The subject compounds and methods find use in a variety of therapeutic applications. Therapeutic applications of interest include those applications in cancer treatment. Of particular interest is treatment of prostate cancer.

### Pharmaceutical Compositions

The herein-discussed compounds can be formulated using any convenient excipients, reagents and methods. In certain embodiments, there is provided a pharmaceutical composition comprising a subject compound and a pharmaceutically acceptable excipient. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the subject compound and a second active agent (e.g., as described herein), e.g. a small molecule, a chemotherapeutic, an antibody, an antibody fragment, an antibody-drug conjugate, an aptamer, or a protein, etc. are administered to individuals in a formulation (e.g., in the same or in separate formulations) with a pharmaceutically acceptable excipient(s). In some embodiments, the second active agent is a chemotherapeutic agent. In certain embodiments the chemotherapeutic agent is a taxane e.g. pacliataxel; or an anti-androgen.

In another aspect of the present invention, a pharmaceutical composition is provided, comprising, or consisting essentially of, a compound of the present invention, or a pharmaceutically acceptable salt, isomer, tautomer or prodrug thereof, and further comprising one or more additional active agents of interest. Any convenient active agents can be utilized in the subject methods in conjunction with the subject compounds. In some instances, the additional agent is a chemotherapeutic agent. The subject compound and chemotherapeutic agent, as well as additional therapeutic agents as described herein for combination therapies, can be administered orally, subcutaneously, intramuscularly, intranasally, parenterally, or other route. The subject compound and second active agent (if present) may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ. In certain cases, the therapeutic agents can be administered intranasally. In some cases, the therapeutic agents can be administered intratumorally.

In some embodiments, the subject compound and a chemotherapeutic agent are administered to individuals in a formulation (e.g., in the same or in separate formulations) with a pharmaceutically acceptable excipient(s). The chemotherapeutic agents include, but are not limited to alkylating agents, nitrosoureas, antimetabolites, antitumor antibiotics, plant (vinca) alkaloids, and steroid hormones. Peptidic compounds can also be used. Suitable cancer chemotherapeutic agents include taxane and active analogs and derivatives thereof; dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

The subject compound and second chemotherapeutic agent, as well as additional therapeutic agents as described herein for combination therapies, can be administered orally, subcutaneously, intramuscularly, parenterally, or other route. The subject compound and second chemotherapeutic agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### EXAMPLES

The following examples are put forth so as to provide those of ordinary skill in the art with a complete disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present disclosure. All such modifications are intended to be within the scope of the claims appended hereto.

### Novel Glycolytic Inhibitor as Treatment of Aggressive Prostate Cancer

Among men, prostate cancer is the most common malignancy and the second leading cause of cancer mortality, with aggressive disease remaining a significant clinical challenge. We describe a molecule, SU086, that inhibits the growth of prostate cancer cells, cell line and patient-derived xenografts, and ex vivo prostate cancer patient specimens. Proteomic analysis indicates SU086 mechanism of action is linked to perturbation of glycolysis. In combination with standard of care second-generation antiandrogen therapies, SU086 has synergistic or additive effects on impairing prostate cancer cell and tumor growth. Our study identifies SU086 as a new treatment for aggressive prostate cancer as single agent or combination strategy.

Herein, we describe a chalcone derivative, SU086, as an anticancer agent for prostate cancer. Proteomic profiling by LC-MS/MS analysis and validation studies implicate SU086 mechanism of action in the impairment of the glycolysis pathway. In context of prostate cancer, SU086 inhibited prostate cancer cell growth, migration and invasion in vitro. Moreover, SU086 significantly delayed the tumor growth of cell line xenograft models of CRPC, hormone-sensitive and resistant patient-derived xenografts (PDXs) in vivo and the proliferation of localized human prostate cancer patient samples ex vivo. Furthermore, SU086 strongly synergized with standard of care second-generation antiandrogens, enzalutamide and abiraterone, in inhibiting prostate cancer cell and tumor growth in vivo. Our study identifies SU086 alone or in combination therapy settings as a unique treatment for aggressive prostate cancer, which is currently incurable. In summation, SU086 is a novel therapeutic which inhibits aggressive prostate cancer growth and is effective in both AR-sensitive and AR-insensitive settings.

### Results

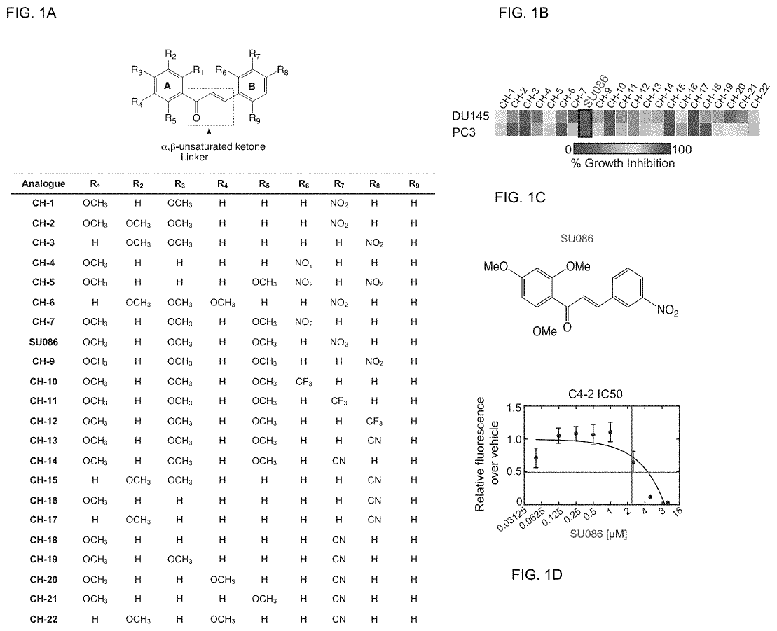

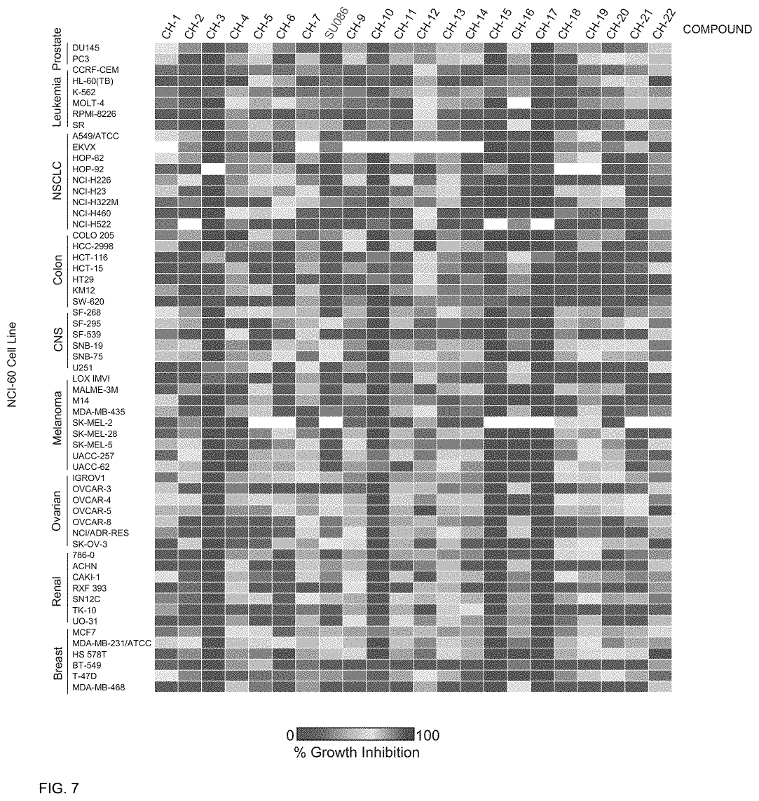

SU086 as a potent inhibitor of prostate cancer cell growth, migration and invasion. We developed a library of chalcone compounds, (FIG. 1a). A selection of twenty-two of these compounds were tested for broad anticancer activity with the National Cancer Institute's 60 human cancer cell line panel (NCI-60 cell line screen) which included two AR-independent prostate cancer cell lines, DU145 and PC3 at an initial dose of 10 μM (FIG. 7). Irrespective of number and position of methoxy (OCH₃) groups on ring A, compounds with cyano (CN) substitution on ring B (CH-13 through CH-22) did not show much growth inhibition in either of the tested prostate cancer cell lines DU145 and PC3, except CH-18, which showed almost complete inhibition in PC3, but only moderate inhibition in DU145 cell line. Compounds with trifluoromethyl (CF₃) substitution on ring B (CH-10-CH-12) were also ineffective in both cell lines (FIG. 1a and FIG. 7a). Interestingly, a trimethoxy-chalcone derivative consisting of nitro (NO₂) group on ring B, SU086, was highly effective against a variety of cancer cell lines and completely inhibited the growth of both prostate cancer cell lines DU145 and PC3 in the NCI panel (FIG. 1a,b, FIG. 7). The majority of treatments for aggressive prostate cancer to date target signaling through AR by inhibition of androgen biosynthesis, sequestration of free androgens, or restricting access of AR translocating to the nucleus, preventing downstream signaling. As DU145 and PC3 are AR-negative prostate cancer cells lines, inhibition by SU086 provides an interesting new strategy for prostate cancer therapies. A previous study of compounds of this library observed impairment of AR nuclear translocation due to inhibition of heat shock proteins which act as AR chaperones, which suggests there may be AR-positive and AR-negative effects of the compound. Thus, in vitro, we tested SU086 on a cohort of prostate cancer cells with an arrayed AR status.

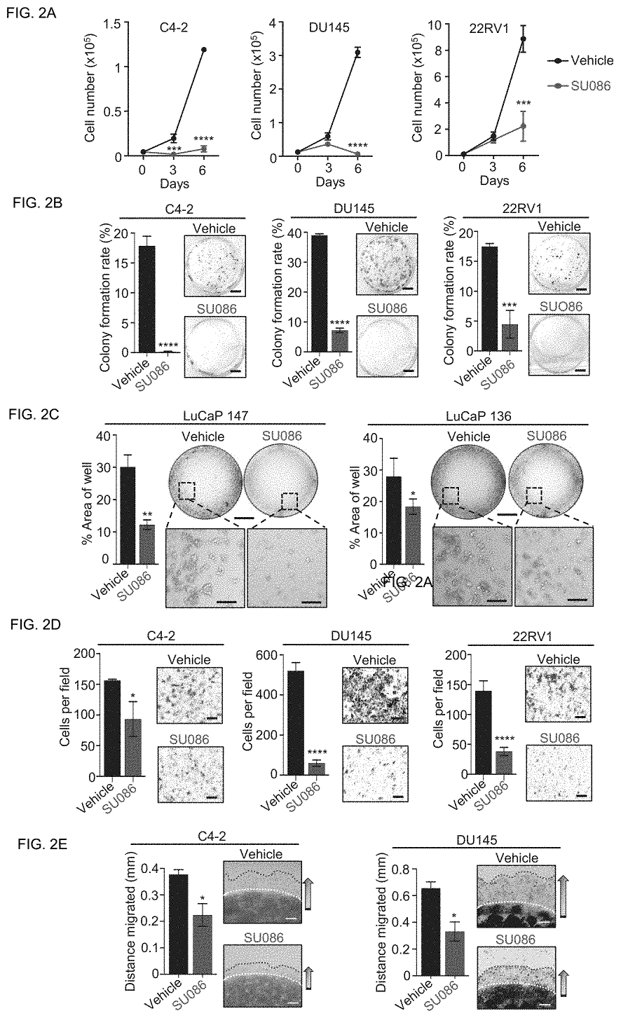

SU086 displayed significant growth inhibition and low toxicity in viability studies, with a final IC₅₀ determined in the CRPC cell line C4-2 of 2.8 μM (FIG. 1c and FIG. 2a). Treatment of cells with SU086 in C4-2 (AR-positive) as well as additional CRPC cell lines, DU145 and 22RV1 (AR-positive with expression of aggressive splice variant, AR-V7) led to a significant inhibition of prostate cancer cell growth, highlighting that our observed effects of SU086 were independent of AR status (FIG. 2a,b).

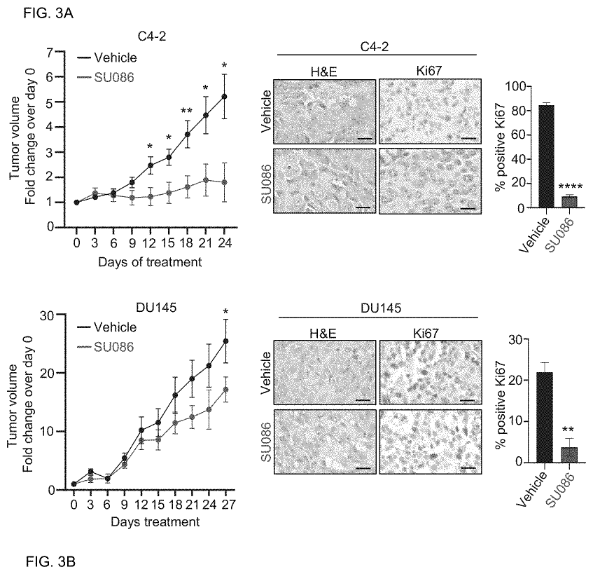

In addition to activity in CRPC cell lines, SU086 had strong growth inhibitory effects on prostate cancer cells derived from two androgen-dependent patient-derived xenografts (PDXs) cells, LuCaP 136 and LuCaP 147. These cells were cultured as tumorspheres and treated with 1 μM SU086 or vehicle control. In both PDX cell lines, SU086 inhibited tumorsphere growth (FIG. 2c). To determine the effects of SU086 on migratory and invasive characteristics of aggressive prostate cancer cells we performed transwell migration chamber experiments (FIG. 2d) and 3D Matrigel Drop invasion assays (FIG. 2e). In both, treatment with SU086 inhibited prostate cancer cell migration and invasion (FIG. 2d-e).

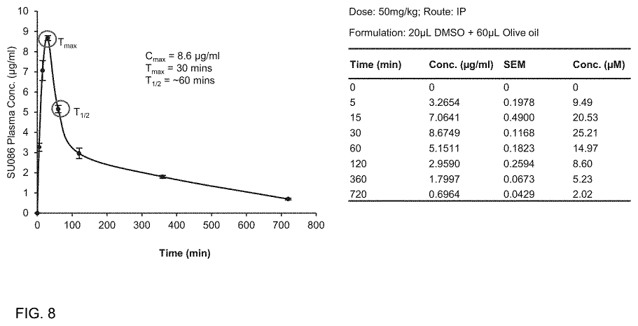

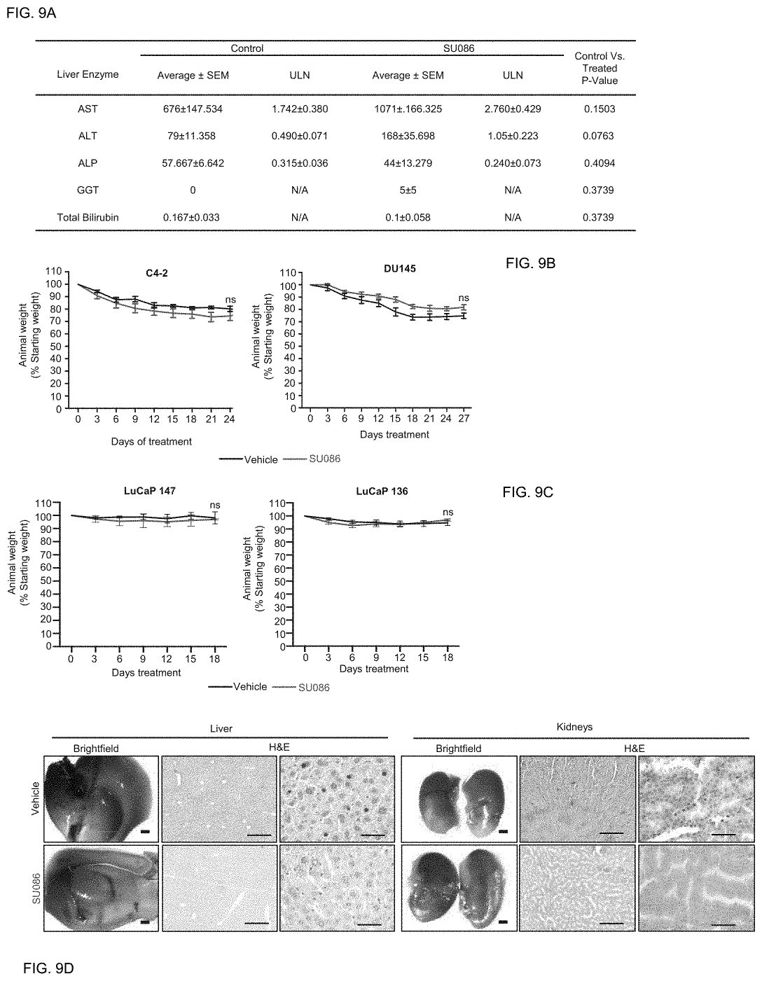

SU086 inhibits prostate cancer growth in preclinical models of prostate cancer in vivo. We next set to examine the therapeutic efficacy of SU086 in prostate cancer in preclinical settings. Pharmacokinetic (PK) and maximum tolerated dose (MTD) analysis of SU086 were performed. Results from PK studies were favorable, although observed half-life was short with a T_{1/2} value of 1 hour (FIG. 8). The MTD was determined at 50 mg/kg, delivered i.p. based on solubility, and was determined to have limited to no measurable toxicity, as evidenced by liver enzyme panel, and health of liver and kidney tissue at this dose (FIG. 9a). SU086 treatment of mice bearing prostate cancer xenografts from C4-2 and DU145 cells resulted in significantly delayed tumor growth in vivo (FIG. 3a,b). Consistent with the liver enzyme panel analysis, we did not observe significant decrease of animal body weight upon treatment with SU086 when compared to the vehicle control groups (FIG. 9b). Immunohistochemical analysis of proliferation marker (Ki67) in all tissues reflected decreased proliferative index after treatment with SU086 (FIG. 3a, b).

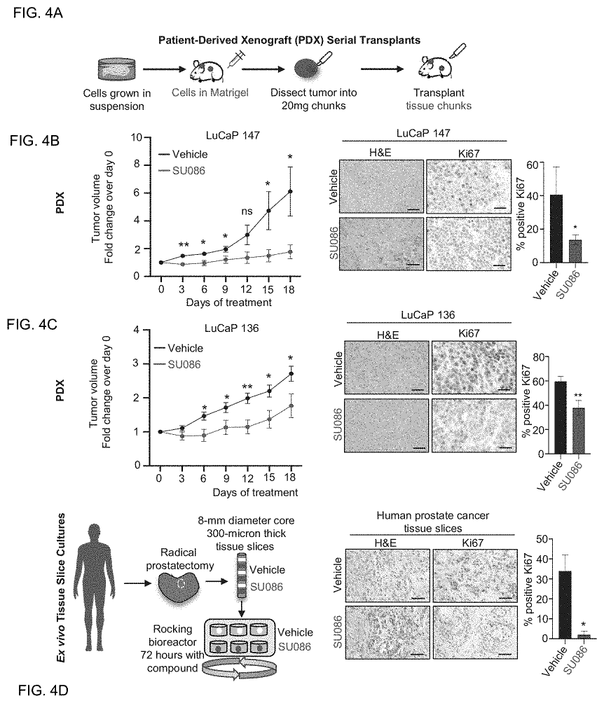

Preclinical efficacy of SU086 was further tested in patient-derived xenograft (PDX) models of prostate cancer. Tumors derived from LuCaP 147 and LuCaP 136 cells were serially propagated in immunodeficient NOD SCID gamma (NSG) male mice with testosterone (FIG. 4a). Mice were randomized into SU086 or vehicle treated groups and treatment initiated when tumors reached 50-mm³. Tumor growth was halted in the case of LuCaP 147 and significantly delayed for LuCaP 136 measured by changes in tumor volumes over time and Ki67 in tumor tissues by proliferative index (FIG. 4b,c). No measurable toxicity was observed assessed by animal body weight upon treatment with SU086 when compared to the vehicle control groups and histology of livers and kidneys (FIG. 9c,d).

To further evaluate the therapeutic potential of SU086, we utilized tissue slice cultures of primary human prostate cancer (Gleason grade 4+3) derived from a radical prostatectomy specimens. Thin, precision cut tissue slices were maintained in a rotating culture apparatus 72 hours in the presence of vehicle control or SU086 (elevated to 5 μM to facilitate diffusion into tissues). The proliferation index, evaluated by immunohistochemical staining with antibody against Ki67, was dramatically reduced from ˜30% to less than 5%, demonstrating anti-proliferative activity of SU086 in primary prostate cancer (FIG. 4c). Together these data demonstrate a robust potential of SU086 as a prostate cancer therapy.

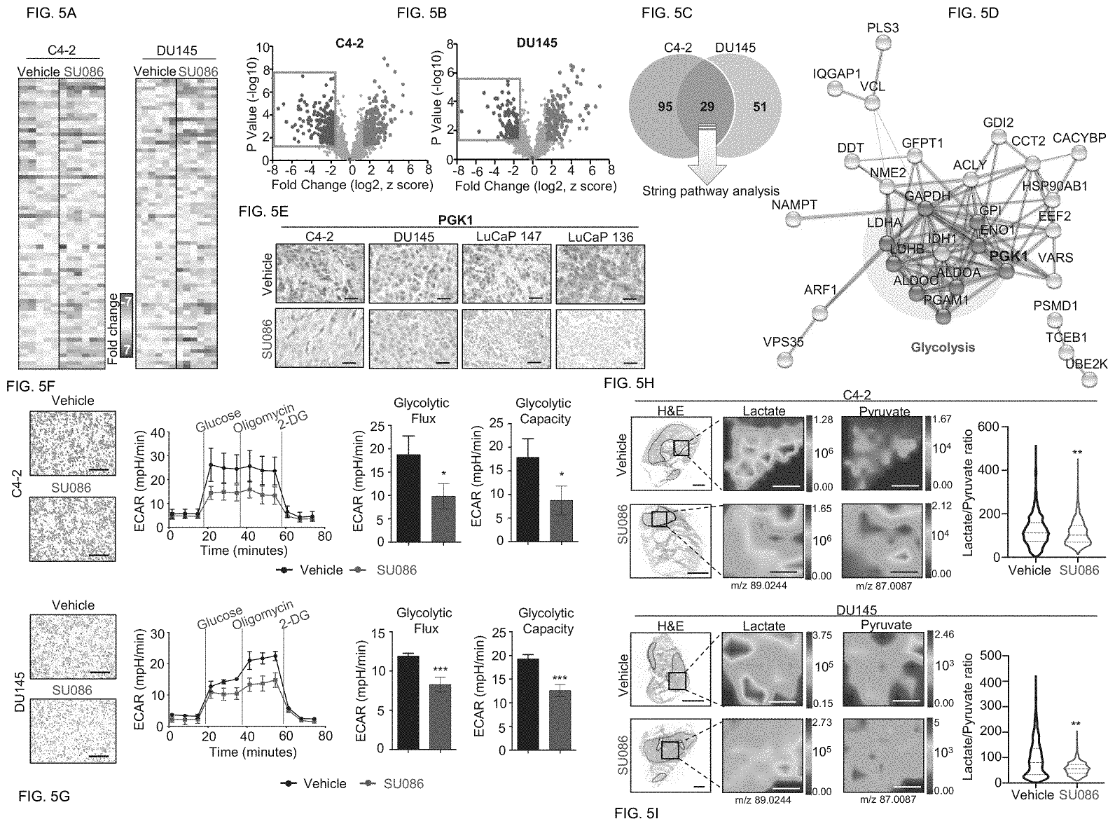

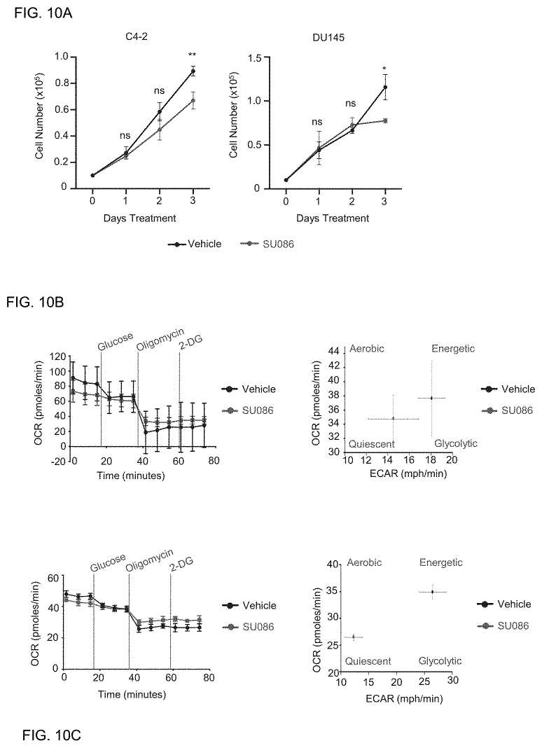

Proteomic profiling reveals that SU086 alters prostate cancer glycolysis. To delineate the mechanism through which SU086 inhibits prostate cancer growth, we evaluated global protein changes upon treatment with SU086. C4-2 and DU145 cells were treated with SU086 at 1 μM for 48 hours, a time point at which cell proliferation was not yet significantly impacted (FIG. 10a). Protein lysates were utilized to perform label-free LC-MS/MS spectral analysis of the total proteome. Twenty-nine proteins were significantly down-regulated by SU086 in both C4-2 and DU145 cells (>2-fold decrease with p value<0.01) (FIG. 5a-c). By mapping protein-protein interaction networks (STRING analysis), we identified an enrichment of glycolytic regulators (FIG. 5d). These observations suggest that the target of SU086 may potentially act upstream of the glycolysis pathway, inhibiting or preventing the glycolytic transition in aggressive prostate cancer. From the enrichment analysis we focused on phosphoglycerate kinase-1 (PGK1), a central enzyme in ATP synthesis, and when active, acts as a shunt from the TCA cycle into glycolysis. PGK1 is commonly expressed in many cancers with associations to metastasis and multidrug resistance. Prostate cancer produces PGK1 which regulates metastasis to the bone, the most common metastatic prostate cancer site. Histological assessment of tissues from C4-2, DU145, LuCaP 147 and LuCaP 136 tumors treated with vehicle or SU086 confirmed that PGK1 protein levels were significantly decreased upon treatment with SU086 (FIG. 5e).

To further assay the downstream effects on glycolysis we performed a Seahorse glycolytic stress test of C4-2 and DU145 cells pretreated for 24 or 48 hours with SU086 or DMSO control (FIG. 5f,g and FIG. 9). Extracellular acidification rate (ECAR), the excretion of H+ ions after metabolic stimulation, is a proxy for glycolytic production of lactate. ECAR levels were measured, as well as oxygen consumption rate (OCR), an indicator of mitochondrial respiration. Glycolytic flux and glycolytic capacity were calculated using ECAR. SU086 decreased both the glycolytic flux (˜50%) and the glycolytic capacity (30-50%) of both cell lines. Conversely, oxygen consumption rates (OCR) did not undergo any changes (FIG. 10b,c). The assayed treatment time point of 24 hours precedes an effect of SU086 on proliferation (FIG. 10a), suggesting the decreased glycolytic performance precedes the effects on proliferation rate, and confirm the effect on glycolytic capacity was not a result of differences in cell number. Another observation in this study suggested evidence of a differing metabolic profile in C4-2 and DU145 cells. Namely, C4-2 cells in a glucose-free environment only required the addition of glucose, and not oligomycin, to reach their maximum glycolytic capacity (FIG. 5f,g). DU145 cells, however, further increased ECAR upon addition of oligomycin and loss of ATP production from mitochondrial respiration. Consistently, DU145 cells are heavily reliant on glucose in metabolism. These results indicate a role for SU086 in the inhibition of glycolytic metabolism.

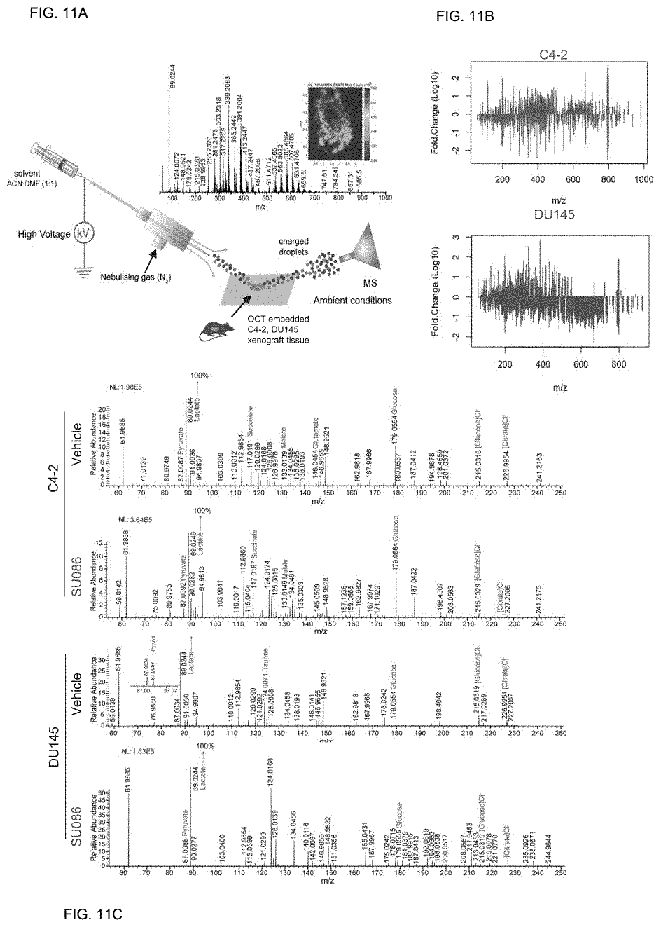

SU086 decreases intratumoralmetabolism. To visualize the active metabolites present in the C4-2 and DU145 prostate cancer xenografts, and discern alterations in the metabolic profiles of those treated with SU086, we performed desorption electrospray ionization-mass spectrometry (DESI-MS) of flash frozen, OCT-embedded tissue (FIG. 11a). After scanning, H&E analysis of the tissue was performed to allow for exclusion of necrotic and blank regions from analysis (FIG. 5h,i). Representative MS spectra from each group is shown in Extended Data FIG. 5b. Healthy tissue regions from two (C4-2) or six (DU145) separate tumors per group were pixelated, with individual metabolic values for every coordinate (FIG. 11b). Statistical analysis of microarrays (SAM) was performed on data to reduce false discovery rates, allowing us to determine statistical significance of changes in metabolites between treatment groups. One such observation includes lactate and pyruvate levels in the xenograft tissue. We observed that lactate and pyruvate levels were altered in xenograft tissues. As glycolysis produces lactate, a higher lactate/pyruvate ratio is indicative of a greater rate of glycolysis. Lactate/pyruvate ratios were decreased in SU086 treated C4-2 and DU145 tumors, consistent with inhibited glycolysis following SU086 treatment (FIG. 5h,i and FIG. 11c).

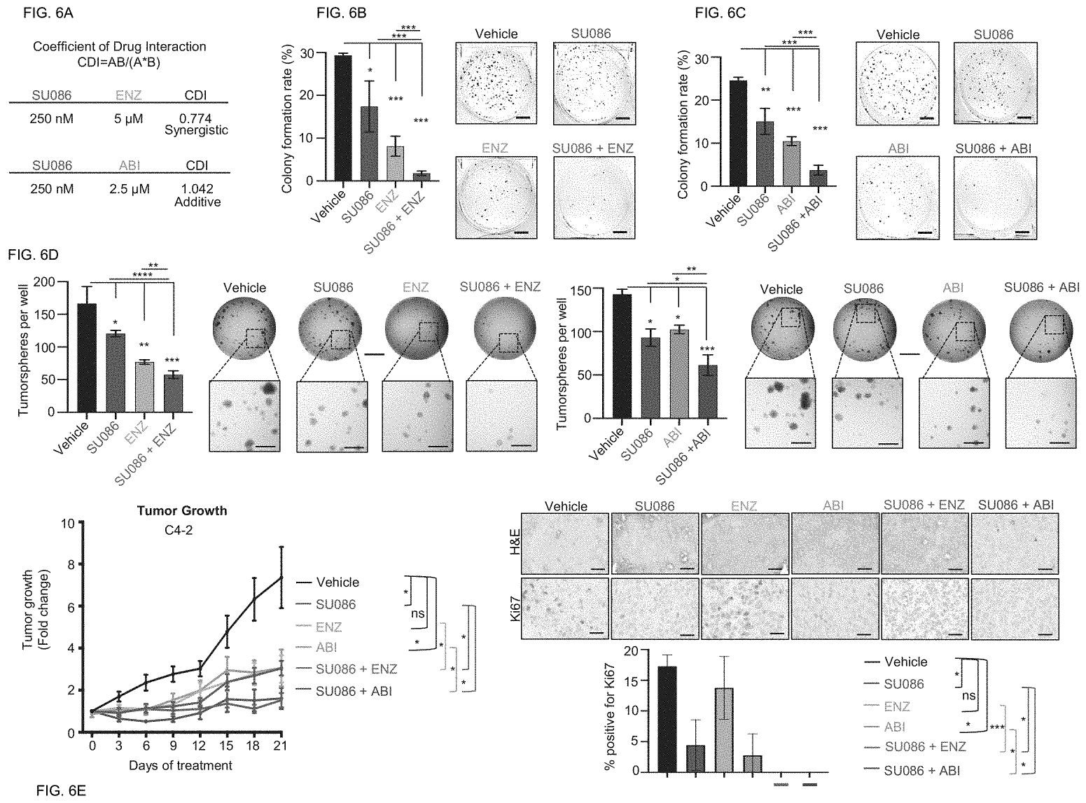

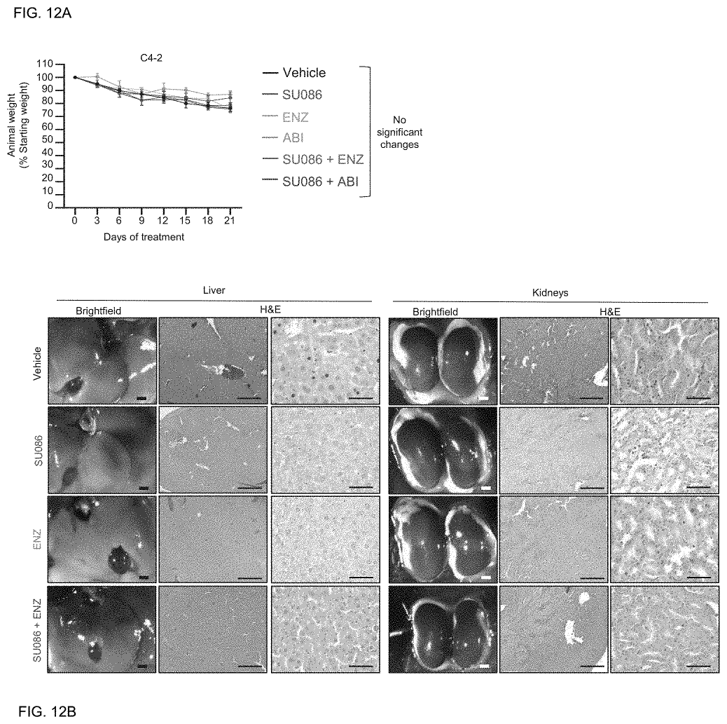

SU086 synergizes with standard of care second generation anti-androgens in inhibiting prostate cancer growth. As the observed effects of SU086 are independent of AR, we explored the potential of a combination strategy with standard of care therapies such as second-generation antiandrogens enzalutamide or abiraterone. C4-2 CRPC cells were chosen for their positive AR status, a necessary target for anti-androgen therapy. Coefficient of drug interaction (CDI) was assayed using colony formation in C4-2 cells. Using CDI=Ratio of combined drugs over control, divided by ratio of drug A over control times ratio of drug B over control, a score<1 indicates synergy, a score=1 indicates additive effects, and a score>1 represents antagonism. Enzalutamide plus SU086 synergized with a score of 0.77, while activity of abiraterone with SU086 was additive, with a score of 1.042. (FIG. 6a) Based on this assay, we used a lower dose of SU086, 250 nM for subsequent combination experiments in vitro. In combination with enzalutamide (ENZ, 5 μM) or abiraterone (ABI, 2.5 μM), SU086 at nanomolar concentration dramatically attenuated colony formation (FIG. 6b). Similarly, SU086 had a large combinatorial effect with ENZ or ABI in inhibition of LuCaP 136 tumorsphere growth (FIG. 6a, b,c). Notably, SU086 needed to remain at the single therapy dose of 1 μM to infiltrate Matrigel. In C4-2 xenografts, SU086 (50 mg/kg, daily i.p.) with ENZ (10 mg/ml, daily gavage) or ABI (200 mg/kg, daily gavage) halted tumor growth throughout the 21 days of treatment (FIG. 6d), and proliferation assessed by Ki67 staining, in combination tumors was negative, consistent with no growth (FIG. 6e). These results indicate that SU086 is a very potent therapeutic strategy when paired with standard of care therapy for deadly CRPC. Furthermore, we did not detect measurable toxicity as assessed by animal body weight and liver and kidney toxicity (FIG. 12).

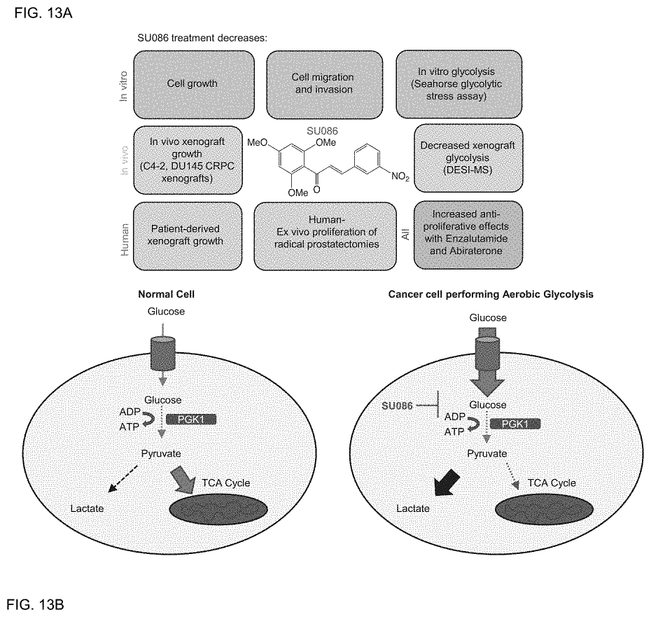

Metabolic reprogramming is one of the hallmarks of cancer. Termed the Warburg effect, the transformation of cancer cells from performing energy efficient oxidative phosphorylation to the less energy producing aerobic glycolysis is a commonly observed phenomenon. However, prostate cancer metabolism is unique in that the Warburg effect is observed only at late stage disease; is associated with castration-resistance and neuroendocrine differentiation, and may be involved in driving these phenotypes. In our tested models, SU086 inhibited prostate cancer oncogenesis through impairment of glycolysis (graphical abstract in FIG. 13). This study provides unique insights into differences in metabolism of prostate cancer tumors, as well as emphasizes the importance of glycolytic metabolism to late stage prostate cancer survival. Consistent with literature, metabolic profiles of late stage prostate cancer, our observed lactate/pyruvate ratios, and evidence from glycolytic stress test, C4-2 are basally more dependent on glycolysis over oxidative phosphorylation. As such, these cells appear to be undergoing the canonical cancer “Warburg effect”. DU145 cells, which are more reliant on glucose production, had a slightly different metabolic response to SU086 (FIG. 5g,i). Interestingly this coincided with C4-2 having greater inhibition by SU086 both in vitro and in vivo, which may be in part attributed to this phenomenon. Other possibilities include differential AR status, as well as PTEN allele status (C4-2 is PTEN⁻/⁻, DU145 PTEN^{+/-}), which could influence PGK1 levels. PGK1 self-phosphorylates, leading to the first ATP-generating step of metabolism. In glioblastoma, PTEN dephosphorylates PGK1, preventing metabolic reprogramming. As PTEN is a tumor suppressor commonly lost in prostate cancer, this observation could further the understanding of the role PTEN loss plays in oncogenic transformation. PGK1 has further been associated with an “angiogenic switch” which may correspond with metabolic reprogramming. PGK1 has been examined as a therapeutic target, as it plays a vital role in cancer metabolism, tumor metastasis and therapy resistance in multiple malignancies.

While SU086 displays promising monotherapeutic potential in our models, clinically, the oncology field is increasingly shifting towards combinatorial treatment strategies to improve overall patient survival for prostate cancer as well as many other malignancies. Inhibition of glycolysis in preclinical studies of prostate cancer has been associated with androgen sensitivity, therefore we assayed SU086 with standard of care enzalutamide or abiraterone treatments and observed synergistic or additive effects, respectively. Canonically, prostate cancer patients, especially in early stage localized disease, have been treated with one modality or therapeutic at a time: androgen-deprivation; radiotherapy; second-generation anti-androgens; chemo- or immuno-therapies. However, prospective prostate cancer clinical trials are experiencing a shift towards combining strategies, with incorporation of multiple standard of care options. One strategy being explored is combined androgen blockade (CAB), with enzalutamide or abiraterone acetate plus ADT or Apalutamide; or abiraterone combined with enzalutamide. These methods are translating to chemotherapies such as Docetaxel and ADT in the CHAARTED trial and radiation therapy with second generation anti-androgens.

Based on our data, SU086 provides a new therapeutic strategy for localized prostate cancer, as well as CRPC. SU086 can also be used as an anticancer agent in other glycolytic cancers. Oncogenic metabolism is highly conserved across cancers. Cancer metabolism has been associated with multi-drug resistance across a variety of cancer and drug classes, therefore targeting cancer metabolism through inhibitors of key glycolytic regulators is a rapidly emerging field. SU086, in addition to the effects on prostate cancer, demonstrated complete growth inhibition in 28/59 (47%), and greater than 90% growth inhibition in 44/59 (74.5%) of cancer cell lines assayed in the NCI-60 panel (FIG. 7). Collectively, these findings indicate SU086 possesses great potential as a single therapeutic in prostate cancer, as well as combination strategy with anti-androgen therapy to treat incurable CRPC, and a novel multi-indication cancer metabolism inhibitor.

### CITATIONS

Siegel, R. L., Miller, K. D. & Jemal, A. Cancer statistics, 2019. CA. Cancer J. Clin. 69, 7-34(2019).
2. Scher, H. I. et al. Increased survival with enzalutamide in prostate cancer after chemotherapy. Supplementary appendix. N. Engl. J. Med. 367, Supplementary appendix (2012).
3. Beer, T. M. et al. Enzalutamide in metastatic prostate cancer before chemotherapy. N. Engl. J. Med. 371, 424-433 (2014).
4. de Bono, J. S. et al. Abiraterone and increased survival in metastatic prostate cancer. N. Engl. J. Med. 364, 1995-2005 (2011).
5. Berthold, D. R. et al. Docetaxel Plus Prednisone or Mitoxantrone Plus Prednisone for Advanced Prostate Cancer: Updated Survival in the TAX 327 Study. J. Clin. Oncol. 26, 242-245 (2008).
6. de Bono, J. S. et al. Prednisone plus cabazitaxel or mitoxantrone for metastatic castration-resistant prostate cancer progressing after docetaxel treatment: a randomised open-label trial. Lancet 376, 1147-1154 (2010).
7. Rathkopf, D. E. et al. Safety and Antitumor Activity of Apalutamide (ARN-509) in Metastatic Castration-Resistant Prostate Cancer with and without Prior Abiraterone Acetate and Prednisone. Clin. Cancer Res. 23, 3544-3551 (2017).
8. Fizazi, K. et al. Darolutamide in Nonmetastatic, Castration-Resistant Prostate Cancer. N. Engl. J. Med. 380, 1235-1246 (2019).
9. Rice, M. A., Malhotra, S. V. & Stoyanova, T. Second-generation antiandrogens: From discovery to standard of care in castration resistant prostate cancer. Front. Neurol. 10, 1-12 (2019).
10. Batovska, D. I. & Todorova, I. T. Trends in utilization of the pharmacological potential of chalcones. Curr. Clin. Pharmacol. 5, 1-29 (2010).
11. Dimmock, J. R., Elias, D. W., Beazely, M. A. & Kandepu, N. M. Bioactivities of chalcones. Curr. Med. Chem. 6, 1125-49 (1999).
12. Kontogiorgis, C. A. & Hadjipavlou-Litina, D. J. Synthesis and antiinflammatory activity of coumarin derivatives. J. Med. Chem. 48, 6400-6408 (2005).
13. Going, C. C. et al. Quantitative Proteomic Profiling Reveals Key Pathways in the Anticancer Action of Methoxychalcone Derivatives in Triple Negative Breast Cancer. J. Proteome Res. 17, 3574-3585 (2018).
14. Zhang, S. et al. A novel chalcone derivative S17 induces apoptosis through ROS dependent DR5 up-regulation in gastric cancer cells. Sci. Rep. 7, 1-13 (2017).
15. Szliszka, E. et al. Chalcones enhance TRAIL-induced apoptosis in prostate cancer cells. Int. J. Mol. Sci. 11, 1-13 (2009).
16. Kim, Y. S. et al. Methoxychalcone inhibitors of androgen receptor translocation and function. Bioorganic Med. Chem. Lett. 22, 2105-2109 (2012).
17. Denmeade, S. R. & Isaacs, J. T. A history of prostate cancer treatment. Nat. Rev. Cancer 2, 389-396 (2002).
18. Knudsen, K. E. & Kelly, W. K. Outsmarting androgen receptor: creative approaches for targeting aberrant androgen signaling in advanced prostate cancer. Expert Rev. Endocrinol. Metab. 6, 483-493 (2011).
19. Moses, M. A. et al. Targeting the Hsp40/Hsp70 chaperone axis as a novel strategy to treat castration-resistant prostate cancer. Cancer Res. canres.3728.2017 (2018) doi:10.1158/0008-5472.CAN-17-3728.
20. Nguyen, H. M. et al. LuCaP Prostate Cancer Patient-Derived Xenografts Reflect the Molecular Heterogeneity of Advanced Disease and Serve as Models for Evaluating Cancer Therapeutics. Prostate (2017) doi:10.1002/pros.23313.
21. Young, S. R. et al. Establishment and serial passage of cell cultures derived from LuCaP xenografts. Prostate (2013) doi:10.1002/pros.22610.
22. Saar, M. et al. Spheroid culture of LuCaP 147 as an authentic preclinical model of prostate cancer subtype with SPOP mutation and hypermutator phenotype. Cancer Lett. 351, 272-280 (2014).
23. Valta, M. P. et al. Spheroid culture of LuCaP 136 patient-derived xenograft enables versatile preclinical models of prostate cancer. Clin. Exp. Metastasis (2016) doi:10.1007/s10585-016-9781-2.
24. Maund, S. L., Nolley, R. & Peehl, D. M. Optimization and comprehensive characterization of a faithful tissue culture model of the benign and malignant human prostate. Lab. Investig. 94, 208-221 (2014).
25. Bernstein, B. E. & Hol, W. G. J. Crystal Structures of Substrates and Products Bound to the Phosphoglycerate Kinase Active Site Reveal the Catalytic Mechanism. Biochemistry 37, 4429-4436 (1998).
26. Sun, W. et al. Cofilin-1 and phosphoglycerate kinase 1 as promising indicators for glioma radiosensibility and prognosis. Oncotarget 8, 55073-55083 (2017).
27. He, Y. et al. PGK1-mediated cancer progression and drug resistance. Am. J. Cancer Res. 9, 2280-2302 (2019).
28. Jung, Y. et al. Expression of PGK1 by Prostate Cancer Cells Induces Bone Formation. Mol. Cancer Res. 7, 1595 LP—1604 (2009).
29. Wood, T. E. et al. A novel inhibitor of glucose uptake sensitizes cells to FAS-induced cell death. Mol. Cancer Ther. 7, 3546-3555 (2008).
30. Rajeshkumar, N. V et al. Therapeutic Targeting of the Warburg Effect in Pancreatic Cancer Relies on an Absence of p53 Function. Cancer Res. 75, 3355 LP—3364 (2015).
31. Bok, R. et al. The role of lactate metabolism in prostate cancer progression and metastases revealed by dual-agent hyperpolarized 13 C MRSI. Cancers (Basel). 11, (2019).
32. Hanahan, D. & Weinberg, R. A. Hallmarks of cancer: the next generation. Cell 144, 646-674 (2011).
33. Pavlova, N. N. & Thompson, C. B. The Emerging Hallmarks of Cancer Metabolism. Cell Metab. 23, 27-47 (2016).
34. Choi, S. Y. C. et al. Targeting MCT4 to reduce lactic acid secretion and glycolysis for treatment of neuroendocrine prostate cancer. Cancer Med. 7, 3385-3392 (2018).
35. Eidelman, E., Twum-Ampofo, J., Ansari, J. & Siddiqui, M. M. The metabolic phenotype of prostate cancer. Front. Oncol. 7, 1-6 (2017).
36. Wu, Z., Conaway, M., Gioeli, D., Weber, M. J. & Theodorescu, D. Conditional expression of PTEN alters the androgen responsiveness of prostate cancer cells. Prostate 66, 1114-1123 (2006).
37. Fraser, M. et al. PTEN deletion in prostate cancer cells does not associate with loss of RAD51 function: implications for radiotherapy and chemotherapy. Clin. Cancer Res. 18, 1015-1027 (2012).
38. Qian, X. et al. PTEN Suppresses Glycolysis by Dephosphorylating and Inhibiting Autophosphorylated PGK1. Mol. Cell 76, 516-527.e7 (2019).
39. Wang, J. et al. A glycolytic mechanism regulating an angiogenic switch in prostate cancer. Cancer Res. 67, 149-159 (2007).
40. Cao, H., Yu, H., Feng, Y., Chen, L. & Liang, F. Curcumin inhibits prostate cancer by targeting PGK1 in the FOXD3/miR-143 axis. Cancer Chemother. Pharmacol. 79, 985-994 (2017).
41. Siddiqui, F. A. et al. Curcumin decreases Warburg effect in cancer cells by down-regulating pyruvate kinase M2 via mTOR-HIF1a inhibition. Sci. Rep. 8, 2-10 (2018).
42. Xiao, H. et al. GLUT1 regulates cell glycolysis and proliferation in prostate cancer. Prostate (2018) doi:10.1002/pros.23448.
43. Rathkopf, D. E. et al. A phase 3 randomized, placebo-controlled double-blind study of ARN-509 plus abiraterone acetate (AA) in chemotherapy-naïve metastatic castration-resistant prostate cancer (mCRPC). J. Clin. Oncol. 33, TPS5071-TPS5071 (2015).
44. Chi, K. N. et al. TITAN: A randomized, double-blind, placebo-controlled, phase 3 trial of apalutamide (ARN-509) plus androgen deprivation therapy (ADT) in metastatic hormone-sensitive prostate cancer (mHSPC). Ann. Oncol. 27, (2016).
45. Fizazi, K. et al. Abiraterone plus Prednisone in Metastatic, Castration-Sensitive Prostate Cancer. N. Engl. J. Med. 377, 352-360 (2017).
46. Kyriakopoulos, C. E. et al. Chemohormonal therapy in metastatic hormone-sensitive prostate cancer: long-term survival analysis of the randomized phase III E3805 chaarted trial. J. Clin. Oncol. 36, 1080-1087 (2018).
47. Smith, M. et al. Addition of radium-223 to abiraterone acetate and prednisone or prednisolone in patients with castration-resistant prostate cancer and bone metastases (ERA 223): a randomised, double-blind, placebo-controlled, phase 3 trial. Lancet Oncol. 20, 408-419 (2019).
48. Zaal, E. A. & Berkers, C. R. The Influence of Metabolism on Drug Response in Cancer. Front. Oncol. 8, 500 (2018).
49. Akins, N. S., Nielson, T. C. & Le, H. V. Inhibition of Glycolysis and Glutaminolysis: An Emerging Drug Discovery Approach to Combat Cancer. Curr. Top. Med. Chem. 18, 494-504 (2018).
50. Stoyanova, T. et al. Activation of Notch1 synergizes with multiple pathways in promoting castration-resistant prostate cancer. Proc. Natl. Acad. Sci. U.S.A 113, (2016).
51. Peehl, D. M. Human Prostatic Epithelial Cells. Culture of Epithelial Cells 171-194 (2002) doi:doi:10.1002/0471221201.ch6.
52. Gatto, L. & Lilley, K. S. MSnbase-an R/Bioconductor package for isobaric tagged mass spectrometry data visualization, processing and quantitation. Bioinformatics 28, 288-289 (2011).
53. Garcla-Marques, F. et al. A Novel Systems-Biology Algorithm for the Analysis of Coordinated Protein Responses Using Quantitative Proteomics. Mol. Cell. Proteomics 15, 1740-1760 (2016).
54. Navarro, P. et al. General Statistical Framework for Quantitative Proteomics by Stable Isotope Labeling. J. Proteome Res. 13, 1234-1247 (2014).
55. Tyanova, S. et al. The Perseus computational platform for comprehensive analysis of (prote)omics data. Nat. Methods 13, 731 (2016).
56. Szklarczyk, D. et al. STRING v11: protein-protein association networks with increased coverage, supporting functional discovery in genome-wide experimental datasets. Nucleic Acids Res. 47, D607-D613 (2019).
57. Eberlin, L. S., Ferreira, C. R., Dill, A. L., Ifa, D. R. & Cooks, R. G. Desorption electrospray ionization mass spectrometry for lipid characterization and biological tissue imaging. Biochim. Biophys. Acta 1811, 946-960 (2011).
58. Eberlin, L. DESI-MS Imaging of Lipids and Metabolites from Biological Samples. in Mass Spectrometry in Metabolomics: Methods and Protocols (ed. Raferty, D.) 299-311 (Springer New York, 2014).
59. Vijayalakshmi, K. et al. Identification of diagnostic metabolic signatures in clear cell renal cell carcinoma using mass spectrometry imaging. Int. J. Cancer n/a, (2019).
60. Team, R. C. R: A language and environment for statistical computing. R Foundation for Statistical Computing, Vienne, Austria. https://www.r-project.org/ (2017).
61. Tusher, V. G., Tibshirani, R. & Chu, G. Significance analysis of microarrays applied to the ionizing radiation response. Proc. Natl. Acad. Sci. 98, 5116 LP—5121 (2001).

### Methods

Cell Lines and Culture. 22RV1 and DU145 cells were obtained from ATCC (Manassas, Va.). C4-2 cells were a gift from Dr. Owen Witte (UCLA). All cell lines were authenticated in 2019 through Stanford Functional Genomics Facility. C4-2, DU145 and 22RV1 cells were cultured in RPMI supplemented with 10% FBS, 1% Penicillin/Streptomycin and 1% L-Glutamine, and incubated at 37° C. with 5% CO₂. Warmed Trypsin/EDTA (0.25%) was used for dissociation. LuCaP 136 and LuCaP 147 cells were a gift from Dr. Donna Peehl. Cells were cultured in ultra-low attachment 6 well plates (Corning) in Stempro hESC media with supplements and bovine serum albumin (BSA) (Thermo Fisher, Carlsbad, Calif.) and 10 nM R1881 (Sigma Aldrich). Thawed cells were additionally supplemented with ROK inhibitor 2 μM of Y-27632 for 24 hours, then changed to normal hESC+R1881 media.

Synthesis of Chalcone derivatives: Thin layer chromatography (TLCs) were run on pre-coated Merck silica gel 60F254 plates and observed under UV light. The products were isolated and purified by crystallization or using a Teledyne ISCO Rf Flash chromatography system with hexanes and ethyl acetate as eluents. The ¹H (400 MHz) and ¹³C (101 MHz), NMR spectra were taken on a Varian 400-MR spectrophotometer using TMS as an internal standard. Chemical shifts (6) are expressed in ppm, coupling constants (J are expressed in Hz, and splitting patterns described as follows: s=singlet; d=doublet; t=triplet; q=quartet; m=multiplet. For the verification of the product and purity analysis, the LC-MS was taken on an Agilent 6490 iFunnel Triple Quadrupole 676 Mass Spectrometer, connected to A 2.1×50 mm Zorbax Eclipse Plus C18 column (Agilent Technologies Inc., Santa Clara, Calif., USA). Water was buffered with 0.1% Formic acid and 4 mM Ammonium formate and used as polar solvent, and Acetonitrile was buffered with 0.1% Formic acid and used as non-polar solvent.

Synthesis protocol and characterization data of chalcone derivatives CH-1 to CH-5 and CH-15 to CH-22 were given in our previous report. The novel compounds studied for NCI-60 screening i.e. CH-6, CH-7, SU086 and CH-9 to CH-14 were synthesized using similar method. Briefly, appropriately substituted acetophenone (1.25 mmol) and lithium hydroxide monohydrate (0.251 mmol) were dissolved in ethanol (5 ml) in a round bottom flask and the mixture was stirred at room temperature for 10 min followed by addition of substituted benzaldehyde (1.27 mmol). The reaction mixture was then stirred at RT and the progress of reaction was monitored by TLC using 25% ethyl acetate/hexanes. Upon completion, the reaction was quenched by pouring into ice cold water. The crude product was filtered and crystallized with hot ethanol. NMR and LC-MS data of new chalcones are given below:

CH-6: Obtained in 67.4% yield as light yellow fluffy solid. ¹H-NMR (400 MHz, DMSO): δ 8.72 (1H, t, 1.6 Hz), 8.37 (1H, d, 7.6 Hz), 8.25 (1H, m), 8.11 (1H, d, 15.6 Hz), 7.84 (1H, d, 15.6 Hz), 7.74 (1H, t, 8 Hz), 7.44 (2H, s), 3.89 (6H, s), 3.75 (3H, s). ¹³C-NMR (100 MHz, DMSO): δ 188.23, 153.38, 148.81, 142.67, 141.81, 137.05, 135.32, 133.05, 130.74, 125.11, 125.09, 123.8, 106.86, 60.66, 56.73. LC-MS (ESI-QQQ): m/z 344.1 ([C18H17NO6+H]⁺ calcd. 344.1). Purity 96.2% (rt 5.407 min).

CH-7: Obtained in 61.2% yield as yellow solid. ¹H-NMR (400 MHz, DMSO): δ 8.02-8.04 (1H, m), 7.93 (1H, d, 7.6 Hz), 7.75 (1H, t, 7.6 Hz), 7.62-7.66 (1H, m), 7.53 (1H, d, 16.0 Hz), 6.92 (1H, d, 16.0 Hz), 6.29 (2H, s), 3.82 (3H, s), 3.72 (6H, s). ¹³C-NMR (100 MHz, DMSO): δ 193.25, 162.76, 158.80, 148.78, 138.79, 134.30, 133.17, 131.33, 130.06, 129.67, 125.18, 110.82, 91.42, 56.26, 55.94. LC-MS (ESI-QQQ): m/z 344.1 ([C18H17NO6+H]⁺ calcd. 344.1). Purity 96.9% (rt 5.173 min).

SU086: Obtained in 67.4% as yellow solid. ¹H-NMR (400 MHz, DMSO): δ 8.49 (1H, t, 0.8 Hz), 8.16-8.22 (2H, m), 7.67 (1H, t, 8 Hz), 7.38 (1H, d, 16.0 Hz), 7.15 (1H, d, 16.0 Hz), 6.30 (2H, s), 3.82 (3H, s), 3.71 (6H, s). ¹³C-NMR (100 MHz, DMSO): δ 193.36, 162.63, 158.71, 148.79, 141.14, 136.84, 134.53, 131.78, 130.80, 124.96, 123.68, 111.30, 91.56, 56.29, 55.94. LC-MS (ESI-QQQ): m/z 344.1 ([C18H17NO6+H]⁺ calcd. 344.1). Purity 96.2% (rt 5.253 min).

CH-9: Obtained in 92% yield as yellow solid. ¹H-NMR (400 MHz, DMSO): δ 8.19 (2H, d, 8.8 Hz), 7.96 (2H, d, 8.8 Hz), 7.34 (1H, d, 16.0 Hz), 7.15 (1H, d, 16.0 Hz), 6.30 (2H, s), 3.82 (3H, s), 3.71 (6H, s). ¹³C-NMR (100 MHz, DMSO): δ 193.07, 162.77, 158.81, 148.40, 140.58, 132.92, 129.96, 124.37, 111.21, 91.57, 56.32, 55.96. LC-MS (ESI-QQQ): m/z 344.1 ([C18H17NO6+H]⁺ calcd. 344.1). Purity 95.5% (rt 5.283 min).

CH-10: Obtained in 71.7% yield as yellow solid. ¹H-NMR (400 MHz, DMSO): δ 8.04 (1H, d, 7.6 Hz), 7.76 (1H, d, 7.6 Hz), 7.70 (1H, t, 7.6 Hz), 7.60 (1H, t, 7.6 Hz), 7.46-7.50 (1H, m), 6.30 (2H, s), 3.82 (3H, s), 3.70 (6H, s). ¹³C-NMR (100 MHz, DMSO): δ 193.37, 162.78, 158.71, 138.32 (d, 3 Hz), 133.51, 133.07, 130.80, 128.83, 127.57 (q, 30 Hz), 126.58 (q, 11 Hz), 124.46 (q, 272 Hz), 110.73, 91.34, 56.23, 55.96. LC-MS (ESI-QQQ): m/z 367.1 ([C19H17F3O4+H]⁺ calcd. 367.1). Purity 97.4% (rt 5.577 min).

CH-11: Obtained in 68.9% yield as light yellow solid. ¹H-NMR (400 MHz, DMSO): δ 8.05 (1H, s), 8.00 (1H, d, 8.0 Hz), 7.73 (1H, d, 8 Hz), 7.61 (1H, t, 8.0 Hz), 7.32 (1H, d, 16.4 Hz), 7.11 (1H, d, 16.4 Hz), 6.30 (2H, s), 3.82 (3H, s), 3.70 (6H, s). ¹³C-NMR (100 MHz, DMSO): δ 193.58, 162.54, 158.63, 141.94, 132.29, 131.16, 130.39, 130.24 (q, 32 Hz), 127.00 (q, 4 Hz), 127.31 (q, 4 Hz), 124.46 (q, 271.0 Hz), 111.35, 91.55, 56.27, 55.93. LC-MS (ESI-QQQ): m/z 367.1 ([C19H17F3O4+H]⁺ calcd. 367.1). Purity 96.2% (rt 5.643 min).

CH-12: Obtained in 86% yield as yellow oil, which turned solid after drying. ¹H-NMR (400 MHz, DMSO): δ 7.91 (2H, d, 8.0 Hz), 7.74 (2H, d, 8.0 Hz), 7.31 (1H, d, 16.4 Hz), 7.11 (1H, d, 16.4 Hz), 6.32 (2H, s), 3.84 (3H, s), 3.73 (6H, s). ¹³C-NMR (100 MHz, DMSO): δ 193.27, 162.66, 158.73, 141.53, 138.97 (d, 2.0 Hz), 131.76, 133.33 (q, 32.0 Hz), 129.52, 126.14, (q, 4 Hz), 124.45 (q, 271.0 Hz), 111.28, 91.57, 56.29, 55.94. LC-MS (ESI-QQQ): m/z 367.1 ([C19H17F3O4+H]⁺ calcd. 367.1). Purity 97.4% (rt 5.677 min).

CH-13: Obtained in 63.9% as light yellow fluffy solid. ¹H-NMR (400 MHz, DMSO): δ 7.82-7.88 (4H, m), 7.28 (1H, d, 16.4 Hz), 7.11 (1H, d, 16.4 Hz), 6.29 (2H, s), 3.81 (3H, s), 3.70 96H, s). ¹³C-NMR (100 MHz, DMSO): δ 193.18, 162.70, 158.76, 141.23, 139.54, 133.16, 132.27, 129.53, 119.04, 112.61, 111.26, 91.57, 56.30, 55.95. LC-MS (ESI-QQQ): m/z 324.0 ([C19H17NO4+H]⁺ calcd. 324.1). Purity 98.8% (rt 5.087 min).

CH-14: Obtained in 77% yield as light yellow solid. ¹H-NMR (400 MHz, DMSO): δ 8.21 (1H, s), 8.01 (1H, d, 8.0 Hz), 7.81-7.84 (1H, m), 7.58 (1H, t, 7.6 Hz), 7.25 (1H, d, 16.4 Hz), 7.10 (1H, d, 16.4 Hz), 6.29 92H, s), 3.81 (3H, s), 3.70 (6H, s). ¹³C-NMR (100 MHz, DMSO): δ 193.37, 162.62, 158.69, 141.21, 136.24, 133.86, 133.25, 132.48, 131.39, 130.51, 118.80, 112.61, 111.32, 91.54, 56.29, 55.94. LC-MS (ESI-QQQ): m/z 324.0 ([C19H17NO4+H]⁺ calcd. 324.1). Purity 98.8% (rt 5.087 min).

NCI 60 Cell Line Screening Methodology. Twenty-two chalcone derivatives were screened by the NCI using the NCI 60 Cell line screen. Single dose screening was performed at a drug concentration of 10.0 μM, using sulforhodamine B assay as per standard NCI protocol. Screening results were presented as heatmap graphs indicating growth inhibition from blue (no growth inhibition) to red (complete growth inhibition) (FIG. 1b and FIG. 7).

Cell Viability. 1×10⁴ cells were plated per well of a 96-well plate. Medium was changed 12 hours later with compound at indicated doses in a total volume of 100 μl. After 48 hours, 20 μl of cell titer blue reagent (Promega, Madison, Wis.) were added to 100 μl reaction. Plate was protected from light and returned to incubation for 2 hours at 37° C. Plates were read at 560/590 wavelength on Tecan Microplate Reader. Experiments were performed in triplicate, and a representative experiment is presented±SD.

Cell Proliferation. 1×10⁴ C4-2, DU145 or 22RV1 cells were plated per well of a 24-well plate in triplicate. The following day, Day 0 cells were counted and medium in remaining wells was changed to drug treatment, and subsequently changed every 72 hours. Cells were counted at 3 and 6 days post-treatment with indicated compounds and doses using trypan blue exclusion assay. Experiments were performed in triplicate, and representative experiment is presented±SD.

Colony Formation Assay. 5×10² C4-2, DU145 or 22RV1 cells were plated per well of a six-well plate in triplicate. Cells were treated with DMSO vehicle control, SU086 (1 μM single dose, or 250 nM combination dose), enzalutamide (5 μM), or abiraterone acetate (2.5 μM), or combination thereof. Enzalutamide and abiraterone acetate were purchased from TargetMol (Wellesley Hills, Mass.). Cells were cultured nine days, with media and compounds changed every third day. Colonies were then fixed with ice cold methanol and stained with 0.01% crystal violet one hour at room temperature. To wash, plates were then submerged in water bath for one hour and air dried. Colonies were counted, and colony formation rate (%) determined, quantified as number of colonies per 500 cells×100 as previously described. Experiments shown are representative of three replicates, ±SD.

Migration. Cell lines were pretreated 72 hours with single agent or combination treatment in normal culture in 6-cm² plates. On the day of plating, Matrigel transwell chambers were incubated at 37° C. in unsupplemented RPMI medium for 2 hours. Cells were trypsinized, washed, and switched to serum-free medium. 1×10⁵ C4-2 or 22RV1 cells, or 5×10⁴ DU145 cells were plated in drug-treated, FBS-negative medium on top of transwell. FBS-supplemented medium plus matching drug concentrations was added to bottom of transwell. Chambers were incubated 20 hours, non-migratory cells (cells in top of chamber) were wiped away with cotton swabs, then chambers were fixed with ice-cold methanol and stained with filtered 0.01% crystal violet for one hour, washed with water and air dried. Wells were imaged on stereomicroscope at 80×, 5 images per well and three wells per condition. Cell number represents average of 5 well images in triplicate. Representative image is presented±SD.

Matrigel dot migration assay. 1×10⁵ C4-2 or DU145 cells were counted and resuspended in 20 μl of Matrigel. Matrigel plus cells were pipetted as a 3-dimensional drop onto a dry well of a 24-well plate. Drops were placed in the incubator 30 minutes to solidify, then medium containing indicated compounds at defined doses were added. Media were changed every third day, and cells were grown 6 days. Distance measured from Matrigel edge was calculated by scanning on Celigo, and is an average of three wells. Each experiment was performed in triplicate and is representative. Error bars are ±SD.

Tumorspheres. 48-well plates were coated with 50% Matrigel/50% LuCaP hESC media and placed in 37° C. incubator to solidify. LuCaP 136 and LuCaP 147 cells were mechanically separated by pipetting. A known volume was counted on hemocytometer to approximate cell number. Approximately 1000 cells were plated in 50% Matrigel/50% hESC supplemented medium on top of basement layer. 250 μl of hESC media containing R1881 and indicated compounds were added after 30 minutes incubation, and media were changed every third day. Wells were imaged on Celigo imager and total area covered was quantified on ImageJ for three wells per condition. Error bars represent±SD.

Animals. In conducting research using animals, the investigators adhere to the laws of the United States and regulations of the Department of Agriculture. Further, all animal studies and procedures have been approved and performed in accordance with Stanford Administrative Panel on Laboratory Animal Care (APLAC), IACUC, as well as the USAMRMC Animal Care and Use Review Office (ACURO). Pharmacokinetic analyses was performed in 6-7 week old Balb/c male mice (Charles River Laboratories). Transplant and xenograft studies were performed in 6-8 week old NSG (NOD-SCID-IL2Rγ-null) mice (Jackson Laboratory, Sacramento, Calif.).

Pharmacokinetics study. Mass quantification analysis was carried out using an Agilent 6490 iFunnel triple quadrupole (QQQ) mass spectrometer equipped with an Agilent 1290 infinity II UHPLC. ZORBAX C18 (Eclipse Plus, 2.1×50 mm, 1.8 μm particle size) was used as analytical UHPLC column. The mobile phase was composed of 50% mixture of water (with 0.1% formic acid and 4 mM ammonium formate) and acetonitrile (0.1% formic acid). The flow rate of mobile phase was set at 0.5 mL/min and column temperature was adjusted at 30° C. The electrospray ionization source was operated in positive ion mode. Mass spectrometer parameters were optimized as: source temperature 550° C., nebulizer gas (nitrogen) 20 psi, ion spray (IS) voltage 5000 V, collision energy 35 V. Multiple reaction monitoring (MRM) method was used for the detection of SU086 and its deuterated internal standard SU086-CD₃ SU086 [M+H]⁺ ions were monitored at m/z 344.1 as the precursor ion, and a fragment at m/z 195.1 as the product ion. For SU086-CD₃ the [M+H]⁺ ions were monitored at m/z 347.1 as the precursor ion and a fragment at m/z 198.1 as the product ion. A standard curve was made using known concentrations of SU086 and SU086-CD₃, which was used to calculate the plasma concentration of SU086 at different time points. SU086 was injected at the dose of 50 mg/kg (in 20 μL DMSO/60 μL olive oil) in each mouse at time zero intraperitonially. Blood was collected retro-orbitally at 5, 15, 30, 60, 120, 360 and 720 minutes after injection in three mice per time point. Blood plasma was separated via centrifugation at 7,000 RPM for 10 minutes. Each plasma sample was mixed with SU086-CD₃ internal standard. 10 μL of plasma was mixed with 190 μL MS-grade acetonitrile and vortexed 30 seconds followed by 5 minutes incubation at RT. Mixture was centrifuged at 11,000 RPM for 15 min at 4° C. and supernatant was collected and further cleaned by re-centrifugation. Each protein-free plasma fraction (n=3, from three independent mouse) was resolved using the HPLC/MS MRM method as described above.

Liver Enzyme Analysis. Mice were treated for 24 days with 50 mg/kg SU086 delivered i.p. in olive oil/DMSO. At the experimental endpoint, blood was collected and centrifuged for serum isolation. Three samples per were condition were analyzed. Samples were analyzed for lipid enzymes at the Stanford School of Medicine Diagnostic Laboratory in the Department of Comparative Medicine. Liver enzymes tested include Aspartate Transaminase (AST), Alanine Aminotransferase (ALT), Alkaline Phosphatase (ALP), Gamma-Glutamyl Transferase (GGT), and Total Bilirubin. Samples are charted as average of Units/Liter (U/L)±SEM. Statistical analysis: P Values were calculated with Student's T-test using the U/L data. Samples were further charted over the upper limit of normal (ULN) by dividing by the following values: AST (388 U/L), ALT (160 U/L), ALP (183). GGT and Bilirubin are not often expressed in normal samples and therefore do not have a ULN value. In the SU086-treated arm, only one of three samples had a value for GGT reflecting the large SEM values.

Xenografts. 1×10⁶ C4-2 or DU145 cells were resuspended with 50 μl Matrigel on ice prior to s.c. injection into rear flank of 8-week old NSG mice (n=8-10). Tumors were established to 50-mm³ (3 days-1 week after implantation) prior to drug administration and animals were randomly sorted into treatment group or control. SU086 (50 mg/kg i.p. in 20 μL DMSO/60 μL olive oil) vehicle control was delivered daily thereafter. Animal weights and tumor volumes were measured every third day using calipers, and calculated as (length×width×height)/2. Dose was determined based on maximum dose of SU086 able to be dissolved in solution, tested for animal toxicity prior to study initiation. In combination therapy, animals were randomized into six groups receiving vehicle treatments, SU086, enzalutamide (10 mg/kg daily oral gavage in 5% DMSO, 30% PEG, 65% H20, final volume of 100 μL), abiraterone acetate (200 mg/kg daily oral gavage in DMSO/corn oil) (TargetMol), or combination of SU086 with enzalutamide or abiraterone.

Patient-derived Xenograft Serial Transplant. Approximately 2.5×10⁵ LuCaP 136 and LuCaP 147 cells were injected into flanks of NSG mice in 100% Matrigel. Concurrently, testosterone pellet (testosterone purchased from Sigma), prepared in the lab from 25 mg pressed testosterone, was implanted subcutaneously at the nape of animal neck. Tumors were grown for one month. After harvest, tumors were cut into tissue chunks in sterile environment. Tumors were washed with PBS containing 20 mg/ml gentamycin for 10 minutes. Tumor chunks were sliced to 25 mg, weighed, and placed in individual sterile tubes with PBS on ice until implant. Tumors were implanted by incision in the rear flank, concurrent with a s.c. testosterone pellet as described above. Tumors were grown up to 50-mm³, or one month. Tumors were measured, then mice randomized into treated or control groups. Tumors and animal weight were measured every three days, and volume calculated as (length×width×height)/2. Fold change tumor volume is graphed as tumor size over treatment Day 0 volume of each individual tumor, ±SEM.

Ex vivo. Fresh tissue cores (8-mm diameter) of prostate cancer from radical prostatectomy specimens were acquired by the Stanford University Department of Urology with approval by Stanford Institutional Review Board and informed consent. Cores were precision-cut to 300-μm thickness in a Krumdieck Tissue Slicer (Alabama Research and Development, Mundford, Ala.). Slices were evaluated by H&E staining to verify the presence of cancer, and Gleason grade of cancer in slices neighboring those used for analysis. Slices were sorted alternatively into the vehicle control or SU086 treatment groups, three slices per group. Slices were transferred with sterile forceps on to titanium mesh inserts in 6-well plates with 2.5 mL of culture medium and three slices per well. Complete PFMR-4A was prepared as previously reported. Plates were incubated at 37° C. with 95% air/5% CO₂ on a rotating platform set at a 30° angle (Alabama Research and Development). Intermittent submersion in the medium caused by the angled rotation facilitates nutrient and gas diffusion throughout the slices, critical for maintaining cell viability over time. Tissue acclimated in untreated media overnight, then replaced with media containing treatment of 5 μM SU086 or vehicle control and changed every 24 hours thereafter. Samples were harvested after 72 hours of treatment, formalin-fixed overnight, and paraffin-embedded.

Histology and Immunohistochemistry. Tissue or tumors were collected, brightfield imaged, fixed overnight in 10% Buffered Formalin, then transferred to 70% ethanol and subsequently processed and paraffin-embedded. Tissues were sliced at 4 microns and transferred to slides on 42° C. water bath. Day of histological analysis, slides were heated 1 hour at 65° C. prior to rehydration. Clarifying reagent was used for de-paraffinization, followed by rehydration at 100%, 95% and 70% ethanol. Antigen retrieval was performed in sodium citrate buffer (10 mM) pH 6.0 at 95° C. for 20 minutes in steamer. Sections were blocked in 2.5% goat serum (Vector Laboratories, CA). Sections were incubated with primary antibodies (Santa Crux Biotechnology-anti-PGK1 sc-130335; anti-Ki67 sc-23900) in humidity chamber overnight at 4° C. Slides were washed in 1×PBS and incubated with secondary mouse HRP (Vector Laboratories, CA) for 1 hour, developed using DAB reagent (DAKO), and counter-stained with hematoxylin.

LC-MS/MS and Proteomic Analysis. Two biological replicates of C4-2 and DU145 cells were treated with 1 μM SU086 or vehicle control for 48 hours. Cells were harvested by scraping and lysed in 2% SDS buffer with protease inhibitor and sonicated. Protein was quantified with micro-BCA assay and 25 pg lysate was used per injection, performed in triplicate for two biological replicates. Per run on LC-MS, resulting raw data files were searched twice using Byonic 2.11.0 software (Protein Metrics, San Carlos, Calif.). Data were run against a Swiss-Prot database reference human proteome (2017; 20,484 entries) including search parameters of trypsin digestion with maximum two missed cleavages, precursor mass tolerance-0.5 Da, and fragment mass tolerance-10 ppm with fixed cysteine carbamidomethylation, variable methionine oxidation and asparagine deamination. False discovery rate of >1% was applied to peptide identifications, and quantitative MS1 spectra were extracted from all peptides using an in house R script based on MSnbase package. Protein abundance changes were analyzed using the Generic Integration Algorithm. Statistical weights were calculated at spectrum level, according to WSPP model. Cumulative distributions were plotted to validate the null hypothesis for spectrum, peptide and protein levels, with final statistical analysis performed in Perseus. Finally, proteins with FDR less than 1%, and an accompanying fold-change>2 were analyzed. Protein fittings were compared between down-regulated C4-2 and DU145 samples, identifying 29 which overlapped. Protein interactions were determined by mapping on String database.

Seahorse Glycolytic Rate Assay. For 24-hour assay, cells (7.5×10³ for C4-2 cells and 12.5×10³ DU145) were plated directly onto Seahorse XFp plates, and treatment of either 1 μM SU086 or DMSO was added 6 hours after attachment to a final volume of 80 μl culture medium. Wells were scanned by Incucyte (Essen BioScience, Ann Arbor, Mich.) and normalized based on cell confluence prior to washing with media for Seahorse assay preparation the day of analysis. Then media were washed away with 2 mM glutamine-supplemented Seahorse RPMI medium, pH 7.4, and placed into a deoxygenated 37° C. incubator for one hour in a final volume of 180 μl of Seahorse media. Glycolytic rate assay kits were purchased from Agilent technologies (Santa Clara, Calif.). Glucose, Oligomycin (Oligo) and 2-DG were resuspended according to manufacturer's recommendations and loaded into wells A, B, and C, respectively on flux cartridge. Flux cartridges and wells were hydrated overnight in deoxygenated incubator prior to running assays. Glucose was injected into the wells after an hour in glucose-free Xf Seahorse media, initiating metabolism. Next, oligomycin was injected, peaking glycolytic capacity of cells, prior to quenching of metabolism with 2-deoxyglucose (2-DG). Probes measured live response to metabolic stimulation of either Extracellular Acidification Rates (ECAR), or Oxygen Consumption Rates (OCR). Analysis of both was performed, averaging data from three triplicate wells. Experiments were performed in duplicate, with representative shown). Glycolytic flux is the ECAR value of glucose treated cells minus baseline (time point 6 minus time point 3). Glycolytic capacity is the ECAR value of oligomycin treated cells minus baseline (time point 9 minus time point 3).

DESI-MSI and SAM analysis. DESI-MSI is an ambient ionization imaging technique carried out at room temperature and atmospheric pressure. Experimental details of tissue imaging by desorption electrospray ionization mass spectrometry imaging (DESI) have been described. Briefly, DESI-MSI was performed in the negative ion mode (−5 kV) from m/z 50-1000, using LTQ-Orbitrap XL mass spectrometer (Thermo Scientific) coupled to a home-built DESI-source and a two-dimensional (2D) motorized stage. C4-2 and DU145 xenograft tissues, from mice treated with vehicle or SU086 (from FIG. 2 experiment), were flash frozen at time of harvest and later embedded in OCT. These OCT-embedded tissues were sliced at 12 microns and stored at −80° C. until analysis was performed. Tissues were raster scanned under impinging charged droplets generated from the electrospray nebulization of a histologically compatible solvent system, 1:1 (vol/vol) dimethylformamide/acetonitrile (DMF/ACN, flow rate 1 μL/min). The electrospray nebulization was performed by using sheath gas nitrogen (N₂, 170 psi) and a high voltage of −5 kV. DESI-MSI of all tissue samples were carried out under identical experimental conditions, such as spray tip-to-surface distance ˜2 mm, spray incident angle of 55°, and spray-to-inlet distance ˜5 mm. The fine tuning of these parameters yielded spatial resolution of DESI-MSI of ˜200 μm, defined by the size of spray spot. The MSI data was acquired using XCalibur 2.2 software (Thermo Fisher Scientific Inc.). Ion intensity images were plotted using MSiReader software (version v1.00), and the raw data from each pixel was extracted for statistical analysis. For molecular analysis, tandem-MS and high mass resolution analyses were performed using the LTQ-Orbitrap XL (Thermo Scientific). Tandem-MS spectra were analyzed, and molecular assignments were compared with databases such as LipidMaps, MassBank, and Metlin. The observed species in the negative ion mode represents mostly deprotonated small metabolites related to the aerobic glycolysis, mitochondrial oxidation (TCA) cycle, and deprotonated lipids including free fatty acids (FAs), fatty acid dimers, phosphatidic acids, ceramides, and glycerophospholipids. The tissue samples after DESI-MSI analysis were subjected to histopathologic evaluation using H&E staining to identify viable tissue and exclusionary necrotic regions. XCalibur raw data files were converted to csv files for statistical analysis. Raw csv data was imported to the R language for further processing. After normalizing the intensity of each metabolite by the total ion current for the corresponding pixel, a nearest neighbor clustering method was used to gather pixel intensities corresponding to the nearest molecular ion peak. Based on this approach, we found 26,860 total molecular ion species across both cell lines from 3648 pixels. Based on DESI-MSI measurements, we studied the metabolic differences in 1900 pixels from C4-2 cell lines (2 tissues control, 2 SU086 treated) and 1748 pixels in DU145 cell lines (6 tissues vehicle control, 6 SU086 treated). To identify which metabolites were altered following treatment of SU086, we separately applied in both xenograft sets the SAM (Significance Analysis of Microarrays) method using the same package in R. Based on filtering the metabolites according to a false discovery rate (FDR) cutoff of 5%, SAM identified 1263 metabolites that were altered in C4-2 xenografts and 1593 metabolites in DU145 xenografts. To ensure interpretability of statistical results, we restricted our analysis to peaks for which tandem-MS and subsequent high mass resolution analysis was performed using the LTQ-Orbitrap XL (ThermoScientific).

Statistics. Student's T-test was used to perform two tailed analysis for assays unless otherwise noted.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the following.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A methoxychalcone derivative having anti-cancer activity.

2. A compound of Formula I: 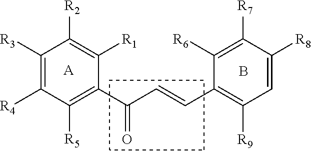
wherein:
R₇ is NO₂; and
each of R₁, R₂, R₃, R₄, R₅, R₆, R₈ and R₉ are independently selected from H, a lower alkyl, an alkoxy group with a lower alkyl; and the like, where a lower alkyl is meant alkyl groups containing from 1 to 6 carbon atoms, usually containing from 1-4 carbon atoms; or a derivative or prodrug thereof.

3. The compounds of claim 1, having the formula: 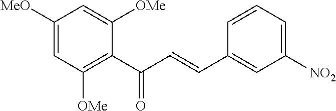 [SMILES: COc1cc(OC)c(C(=O)/C=C/c2cccc([N+](=O)[O-])c2)c(OC)c1]
or a derivative or prodrug thereof.

4. A formulation comprising a compound of claim 2, and a pharmaceutically acceptable excipient.

5. A formulation of claim 4, in a unit dose formulation.

6. A method for treatment of cancer, the method comprising administering to an individual in need thereof and effective amount of a compound of claim 2.

7. The method of claim 6, wherein the cancer is a solid tumor.

8. The method of claim 6, wherein the cancer is prostate cancer.

9. The method of any of claim 6, further comprising administering one or more additional anti-cancer treatments, where the combination may be additive or synergistic.

10. The method of claim 8, wherein the additional anti-cancer treatment comprises administering an anti-androgen compound.
